(19) United States
(12) Patent Application Publication
(10) Pub. No.: US 2011/0029085 A1
(43) Pub. Date: Feb. 3, 2011

(54) FLEXIBLE SPINAL IMPLANT

(75) Inventors: Richard A. Hynes, Melbourne Beach, FL (US); Keith E. Miller, Germantown, TN (US); Virginia L. Richardson, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/605,145

(22) Filed: Oct. 23, 2009

Related U.S. Application Data
(63) Continuation-in-part of application No. 12/533,877, filed on Jul. 31, 2009.

(51) Int. Cl. A61F 2/44

(52) U.S. Cl. 623/17.16

(57) ABSTRACT

A flexible spinal implant for insertion into an intervertebral disc space for sagittal and/or coronal intervertebral stabilization is provided comprising a flexible implant which enables bending of the flexible implant to facilitate insertion of the flexible spinal implant into the disc space via a spinal surgical procedure. The flexible spinal implant comprises a leading end, a trailing end flexibly connected to the leading end, a locking mechanism, wherein the implant is deformable at or about a flexible section to thereby permit a substantially straight entry of the implant into the disc space, and delivered to the selected disc space at a desired insertion angle of approach via a spinal surgical procedure. The implant can have a leading end comprising a curved or bullet shaped configuration, and the flexible section may be comprised of a flexible material or flexible sections which may be lockingly engaged.

### CROSS-REFERENCE TO RELATED APPLICATION

This application is a continuation-in-part of U.S. patent application Ser. No. 12/533,877 filed on Jul. 31, 2009, which is incorporated herein by reference in its entirety.

### FIELD OF THE INVENTION

The present invention relates to medical devices such as spinal intervertebral implants implanted between adjacent vertebral bodies of a spinal column section, and more particularly to a medical implant for intervertebral stabilization comprising a flexible implant section which enables bending or pliancy of the implant body to thereby facilitate insertion of the spinal implant at a selected disc space via a spinal surgical procedure.

### BACKGROUND

The spine is divided into four regions comprising the cervical, thoracic, lumbar, and sacrococcygeal regions. The cervical region includes the top seven vertebral bodies or members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The sacrum region includes five fused vertebral members S1-S5, with S1 being adjacent to L5. The vertebral members of the spine are aligned in a curved configuration that includes a cervical, thoracic and lumbosacral curve. Within the spine, intervertebral discs are positioned between the vertebral members and permit flexion, extension, lateral bending, and rotation. An intervertebral disc functions to stabilize and distribute forces between vertebral bodies. The intervertebral disc is comprised of the nucleus pulposus surrounded and confined by the annulus fibrosis.

Intervertebral discs and vertebral members are prone to injury and degeneration. Damage to the intervertebral discs and/or vertebral members can result from various physical or medical conditions or events, including trauma, degenerative conditions or diseases, tumors, infections, disc diseases, disc herniations, aging, scoliosis, other spinal curvature abnormalities or vertebra fractures. Damage to intervertebral discs can lead to pain, neurological deficit, and/or loss of motion. Damaged intervertebral discs may adversely impact the normal curvature of the spine, and/or lead to improper alignment and positioning of vertebrae which are adjacent to the damaged discs. Additionally, damaged discs may lead to loss of normal or proper vertebral spacing.

Various known surgical procedures, treatments and techniques have been developed to address medical problems associated with damaged or diseased intervertebral discs. One treatment is a fusion procedure which partially removes the center or nuclear area of a damaged disc and fuses adjacent vertebral members to prevent relative motion between the adjacent vertebral bodies. A section of the disc, annulus and nucleus, is removed or cut out to allow insertion of a spinal implant or spacer. The spacer may be used in conjunction with bone graft or allograft material which enables the adjacent vertebrae to grow and fuse together. Existing spinal implants assist in maintaining disc space height during the fusion process while at the same time, permitting or enabling an element of compression and selective movement of the implant within the disc space while vertebral fusion is taking place. The implant or spacer may also assist in imparting desired alignment or lordosis of the adjacent vertebral bodies.

As is known to persons of skill in the art, there are a variety of structures and configurations which can be used to obtain the desired vertebral body spacing and alignment such as spacers, implants or cages. These structures come in a variety of configurations, features, contours, geometries and sizes depending on the specific medical application or use. Further, implants can be inserted from a variety of insertion approaches, including for example anterior, posterior, anterolateral, lateral, direct lateral and translateral approaches.

In the area of surgical procedures for spinal implants at the L4-L5 or the L5-S1 level, an implant is often inserted in the disc space via either an anterior or posterior approach. Delivery and insertion of a spinal implant into the L4-L5 or L5-S1 disc space via a lateral approach can be done, but is less common and more difficult to perform than other procedures such as anterior or posterior procedures. One reason for the difficulty in inserting an implant at the L4-L5 or L5-S1 level via a later approach surgical procedure is the anatomical position of the iliac crest relative to the position of the L4-L5 or L5-S1 disc space level.

The anatomical position and curved nature of the iliac crest relative to the vertebral disc space at L4-L5 or L5-S1 makes the iliac crest a physical obstruction to direct or straight access to the L4-L5 or L5-S1 disc space in a lateral surgical approach procedure. The iliac crest's position prevents a direct or straight angle of approach for delivery, entry and insertion of a spinal implant into the L4-L5 or L5-S1 vertebral disc space. Additionally, at the L4-L5 or L5-S1 disc space levels, as well as higher lumbar spine levels, there is a complexity of neurological and vascular structures that cross the implant delivery approach path or implant path of insertion. In order to clear the obstructing iliac crest, and neurological and vascular structures, for implant insertion at L4-L5 or L5-S1, via a lateral or direct lateral approach, the implant is typically delivered to the disc space at some angled lateral angle of approach.

An additional difficulty in a lateral approach procedure is that since an implant is delivered at some lateral angle of approach, the implant being inserted arrives at L4-L5 or L5-S1 in an angled orientation. It would be easier and more convenient for the implant to enter the disc space in as nearly a direct or straight lateral approach as possible. In order to do this, an implant being inserted into the disc space will have to turn or navigate a corner at the entry of the L4-L5 or L5-S1 disc space so that the implant can enter the disc space in a substantially lateral approach orientation. A drawback of existing implants is that many are rigid or have inflexible physical configurations which prevent the implant from being able to be turned or navigated around a corner. The rigid aspect of existing implant configurations makes it difficult to use or impractical to insert these rigid implants via a lateral approach procedure at L4-L5 or L5-S1. Such difficulties limit the number of lateral approach implant procedures at L4-L5 or L5-S1 and the number of surgeons who can perform such a lateral approach implant procedure.

There is a need for an improved intervertebral implant, and method for inserting an implant between adjacent vertebral bodies using minimally invasive surgical techniques, that overcomes drawbacks and difficulties in delivering and inserting an implant at a desired or selected disc space via a spinal surgical procedure.

### SUMMARY

There is provided a spinal implant for insertion into an intervertebral disc space for intervertebral stabilization comprising a flexible implant section which enables bending or pliancy of the implant body to thereby facilitate insertion of the spinal implant into the disc space via a spinal surgical procedure.

There is provided a spinal implant for insertion into an intervertebral disc space comprising a flexible implant section which enables bending or pliancy of the implant body to thereby facilitate insertion of the spinal implant via a spinal surgical procedure, including, among others, a direct lateral lumbar interbody fusion (DLIF) procedure, a posterior lumbar interbody fusion (PLIF) procedure or a transforaminal lumbar interbody fusion (TLIF) procedure.

There is also provided a spinal implant for insertion into a disc space comprising a leading end, a trailing end and a flexible section connecting the leading end and the trailing end, wherein the implant is deformable at the flexible section to thereby permit a substantially straight lateral entry of the implant into a selected disc space. The implant is delivered to the selected disc space at an insertion angle of approach. The implant can have a leading end comprising a bullet shaped configuration. Further, the flexible section may be comprised of flexible or pliant material, at least one pivoting connection or a spring mechanism.

There is further provided a spinal implant for insertion into a selected disc space comprising, a leading end, a trailing end, a flexible section connecting the leading end and the trailing end and a central implant aperture bounded by the leading end, the trailing end and the flexible section. In one aspect, the implant is delivered at a lateral insertion angle of approach via an implant insertion channel. The implant is deformable or pliant about the flexible section through interaction with the implant insertion channel to thereby permit substantially straight lateral entry of the implant into the selected disc space via a lateral approach. Further, the flexible section may be comprised of flexible or pliant material, at least one pivoting connection or a spring mechanism.

There is also provided a spinal implant for insertion into an intervertebral disc space comprising a leading end, a trailing end flexibly connected to the leading end and a locking mechanism adapted to lockingly engage the spinal implant to prevent motion between the leading end and trailing end. The implant is deformable about a flexible section to thereby permit a substantially straight entry of the implant into a selected disc space. The locking mechanism comprises a leading end locking passage, a trailing end locking passage which substantially aligns with the leading end locking passage when the implant is inserted in the selected disc space, and a locking member adapted to be inserted into the leading end locking passage and trailing end locking passage. The locking mechanism is engaged when the locking member spans the flexible section at a pivotal connection and is at least partially inserted into both the substantially aligned leading end locking passage and trailing end locking passage thereby preventing motion between the leading end and the trailing end. The locking member is secured in both the aligned leading end locking passage and trailing end locking passage through a friction fit or interference fit. The locking member is adapted to cooperatively deform to facilitate insertion of the locking member into the aligned leading end locking passage and trailing end locking passage. The leading end locking passage, the trailing end and the locking member have complimentary and cooperative configurations to enable the locking member to be inserted into the aligned leading end locking passage and trailing end locking passage. Moreover, the leading end and trailing end can form a front to back wedge configuration which is adapted to alleviate coronal spinal deformity when the spinal implant is inserted in the selected disc space. Alternatively, the leading end and trailing end can form a lateral side-to-side wedge configuration which is adapted to alleviate sagittal spinal deformity when the spinal implant is inserted in the selected disc space.

There is further provided an implant system for insertion into an intervertebral disc space comprising a spinal implant deformable about a pivotal connection to thereby permit a substantially straight entry of the implant into a selected disc space. The spinal implant comprises a leading end including a leading end locking passage, a trailing end pivotally connected to the leading end, and having a trailing end locking passage which substantially aligns with the leading end locking passage when the implant is inserted into a selected disc space. The implant system also comprises a locking member adapted to be inserted into the leading end locking passage and trailing end locking passage to lockingly engage the spinal implant to prevent motion between the leading end and trailing end. In locking engagement, the locking member spans the pivotal connection and is at least partially inserted into both the substantially aligned leading end locking passage and trailing end locking passage thereby preventing motion between the leading end and the trailing end. The locking member is secured in both the aligned leading end locking passage and trailing end locking passage through a friction fit or interference fit. Also, the locking member is adapted to cooperatively deform to facilitate insertion of the locking member into the aligned leading end locking passage and trailing end locking passage.

Disclosed aspects or embodiments are discussed and depicted in the attached drawings and the description provided below.

### BRIEF DESCRIPTION OF THE DRAWINGS

FIG. 1 illustrates an anterior view of a section of a vertebral column, the sacrum and ilium;

FIG. 2 illustrates an anterior partial view of FIG. 1 showing a partial insertion of a flexible spinal implant at disc space L5-S1 according to one embodiment of the present disclosure;

FIG. 3 illustrates a side view of a flexible spinal implant according to one embodiment of the present disclosure;

FIG. 4 illustrates a side view of the flexible spinal implant of FIG. 2;

FIG. 5A illustrates an isometric view of a flexible spinal implant according to another embodiment of the present disclosure;

FIG. 5B illustrates a side view of the flexible spinal implant of FIG. 5A in an implant insertion channel according to one embodiment of the present disclosure;

FIG. 5C illustrates an isometric view of a flexible spinal implant according to another embodiment of the present disclosure;

FIGS. 6A-6E illustrate side views of flexible implant sections according other embodiments of the present disclosure;

FIG. 7 illustrates an isometric view of a flexible spinal implant according to another embodiment of the present disclosure;

FIG. 8A illustrates an isometric view of a flexible spinal implant according to another embodiment of the present disclosure;

FIG. 8B illustrates a side view of the flexible spinal implant of FIG. 8A;

FIG. 8C illustrates a front view of the flexible spinal implant of FIG. 8A;

FIG. 9A illustrates an isometric view of a flexible spinal implant according to another embodiment of the present disclosure;

FIG. 9B illustrates a side view of the flexible spinal implant of FIG. 9A;

FIG. 9C illustrates a front view of the flexible spinal implant of FIG. 9A; and

FIG. 10 illustrates a side view of a flexible spinal implant and locking mechanism according to another embodiment of the present disclosure.

### DETAILED DESCRIPTION

The present invention relates to medical devices such as spinal intervertebral implants implanted between adjacent vertebral bodies, and methods of use, and more particularly to a spinal implant for intervertebral stabilization of a spinal disc space via insertion of a flexible or pliant implant at a desired disc space. For purposes of promoting an understanding of the principles of the invention, reference will now be made to one or more embodiments or aspects, examples, drawing illustrations, and specific language will be used to describe the same. It will nevertheless be understood that the various described embodiments or aspects are only exemplary in nature and no limitation of the scope of the invention is thereby intended. Any alterations and further modifications in the described embodiments or aspects, and any further applications of the principles of the invention as described herein are contemplated as would normally occur to one skilled in the art to which the invention relates.

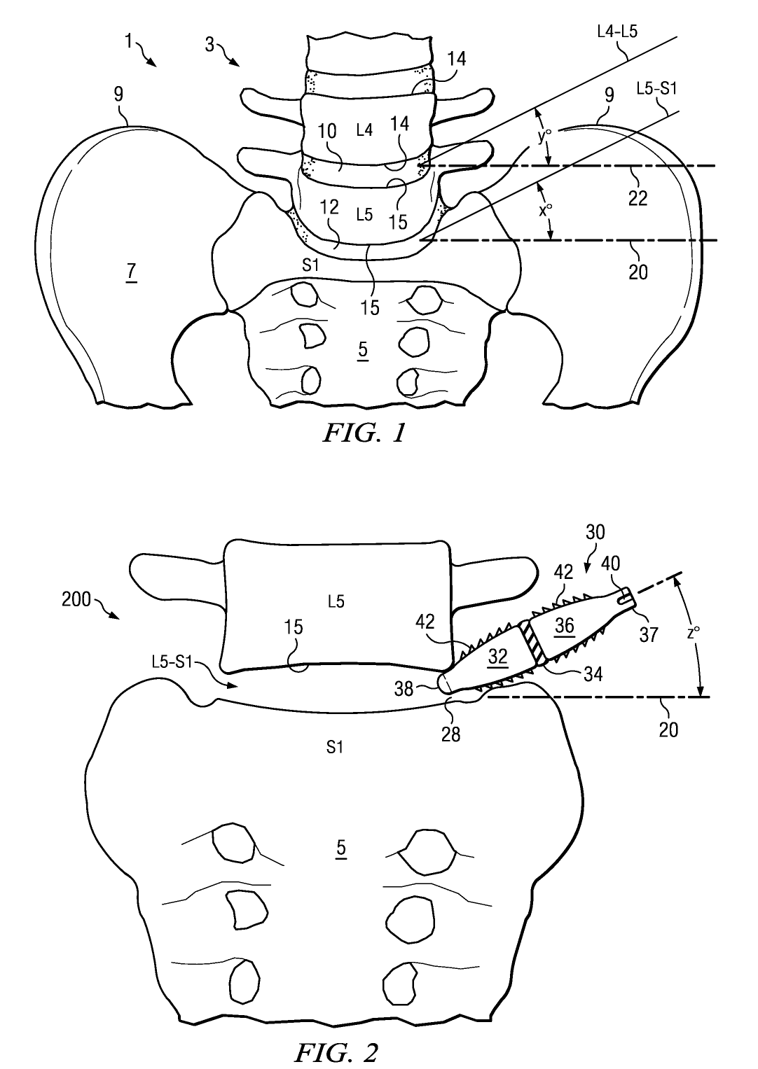

FIG. 1 shows an anterior view of a partial spinal section 1 of the vertebral column 3, the sacrum 5, ilium 7 and iliac crest 9. Also, shown are vertebral bodies L4, L5, Sacrum vertebrae S1, L4-L5 and L5-S1 vertebral disc spaces and the corresponding vertebral discs 10 and 12. The vertebral bodies L4 and L5 include end plates 14 and 15, respectively. FIG. 1 also shows straight or direct lateral reference lines 20 and 22 corresponding to the L5-S1 and L4-L5 disc spaces associated with a lateral approach procedure or lateral fusion surgical procedure. One lateral fusion surgical procedure for inserting an implant is known as a direct lateral interbody fusion (DLIF) procedure.

While FIGS. 1 and 2 illustrate a lateral approach surgical procedure, the flexible spinal implants contemplated and shown in FIGS. 1-10 may also be delivered and inserted into a desired disc space via other spinal surgical approaches and procedures as may be appropriate or required by a patient's anatomy or by a physician. For example, in one preferred aspect, the flexible spinal implant of the present disclosure can be delivered and inserted into a desired disc space via a lateral approach procedure such as a direct lateral lumbar interbody fusion (DLIF) procedure to clear the obstructing iliac crest, and neurological and vascular structures. And, in another preferred aspect, the flexible spinal implant may be delivered and inserted into a desired disc space via a posterior lumbar interbody fusion (PLIF) or transforaminal lumbar interbody fusion (TLIF) procedure to bend around and safely bypass or clear the cauda equina. In one preferred aspect, the flexible spinal implant is delivered via or through a minimal access spinal technology (MAST) surgical technique or procedure. Those of skill in the art will recognize that the flexible spinal implant may also be delivered and inserted via other known surgical approaches, including, a posterior, direct lateral, translateral, posterolateral, or anterolateral or any suitable oblique direction. Some known techniques and approaches that may be used to insert the flexible implant may also include, among others, anterior lumbar interbody fusion (ALIF). Further, those of skill in the art will recognize that a spinal implant may be delivered and inserted through other known surgical technique and procedures, including: open, mini-open or other minimally invasive surgical (MIS) techniques.

Referring to FIG. 1, in a lateral approach procedure, the physical position and configuration of the iliac crest 9 obstructs or prevents a direct or straight line 20 and 22 surgical approach to the L4-L5 or L5-S1 vertebral disc spaces for delivery and insertion of a spinal implant into the disc space. In order to overcome this drawback, and be able to laterally insert the implant at the L4-L5 or L5-S1 disc space via a lateral approach, the implant is delivered to the disc space at a lateral angle of approach, X or Y, relative to the straight lateral reference line 20 and 22. The lateral angle of approach X or Y for implant delivery is selected by a surgeon so as to clear or by-pass the obstructing iliac crest 9 encountered in a lateral approach procedure. Those of skill in the art will recognize that the lateral angle of approach Y corresponding to the L4-L5 disc space may be the same or different than the lateral angle of approach X corresponding to the L5-S1 disc space due to the different disc space positions relative to the iliac crest 9.

FIG. 2 shows an anterior partial view depicting a partial insertion of a flexible spinal implant 30 at disc space L5-S1 according to one aspect of the present disclosure. FIG. 4 also shows the flexible spinal implant 30 of FIG. 2. The flexible spinal implant 30 comprises a leading end 32, a trailing end 36 and a flexible section 34 connecting the leading end 32 and the trailing end 32. The flexible implant 30 also comprises anti-back out protrusions 42 on the upper and lower surfaces 50, 52, 54 and 56 of the flexible implant 30, and an instrument attachment section 40. Those of skill in the art will recognize that the anti-back out protrusions 42 extending from the upper and lower surfaces 50, 52, 54 and 56 will be configured and oriented so as to prevent the implant 30 from backing out or being ejected after implant insertion into the disc space. In the aspect shown in FIGS. 2 and 4, the anti-back out protrusions 42 have a triangular or pyramid configuration and are slanted or oriented back toward the trailing proximal implant end 37 of the implant 30.

The leading end 32 has a physical shape or physical configuration adapted to facilitate or ease implant insertion into the disc space L5-S1. In a preferred aspect, shown in FIG. 2, the leading end 32 has a curved or bullet shaped surface 38 which facilitates insertion of the flexible implant 30 in the L5-S1 disc space. The curved or bullet shaped nose 38 of the leading end 32 may, if the disc space is collapsed, impart a self-distracting force between the L5-S1 disc space which facilitates the insertion of flexible implant 30 into the L5-S1 disc space.

The trailing end 36 of the flexible implant 30 preferably comprises an implant grip or attachment section 40 situated at the proximal implant end 37 which enables the coupling of an insertion instrument (not shown). The attachment section 40 enables the controlled delivery of the flexible implant 30 into the L5-S1 disc space via a lateral surgical approach. In a preferred aspect, the attachment section 40 is recessed into the trailing end 36 such that when an instrument (not shown) is coupled to the flexible implant 30, the instrument is entirely interior to or flush with the exterior surface of the proximal implant end 37. In one aspect, the attachment section is a recessed slot 40 on both sides of the proximal implant end 37.

The flexible section 34 preferably connects the leading end 32 and the trailing end 36 to form the flexible spinal implant 30. The flexible section 34 is preferably located at a mid portion of the flexible spinal implant 30. However, those of skill in the art will recognize that the flexible section 34 may be located at other positions of the flexible spinal implant 30 as may be desired or required by a physician or patient anatomy, or the needs or requirement of a medical procedure. The flexible section 34 is coupled or attached between the leading end 32 and the trailing end 36 so as to form a single assembled flexible spinal implant 30. The flexible section 34 is the aspect that permits or enables the implant 30 to bend, flex or pivot at or about the implant's mid portion when the implant is being inserted into the L5-S1 disc space via a preferred later surgical approach.

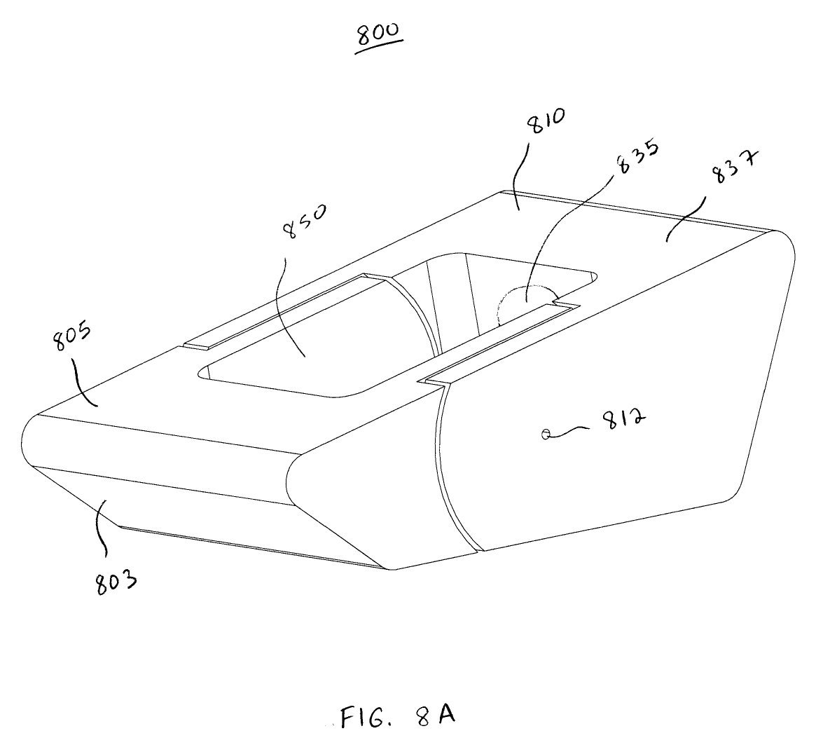

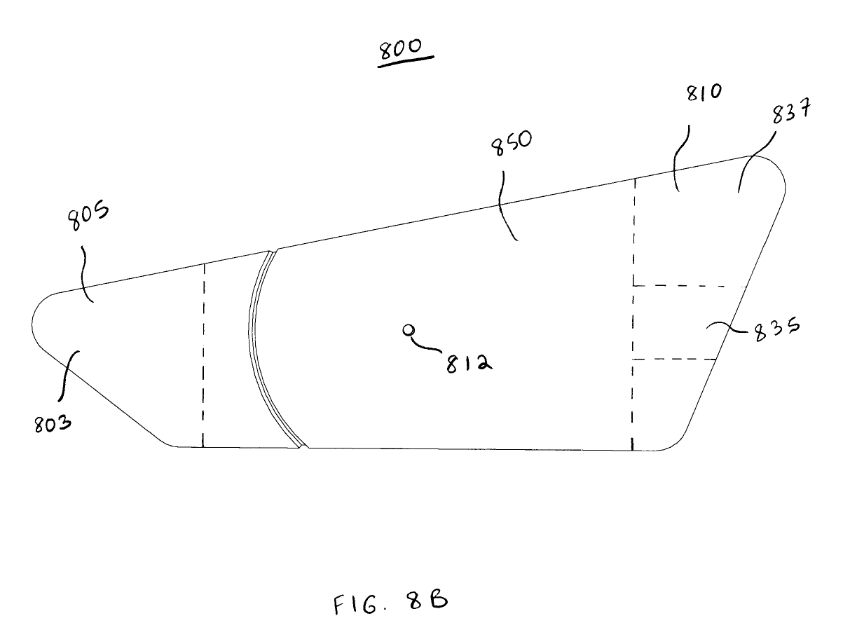

The flexible section 34 also permits the implant 30 to be fully flexible in any one or more dimensional directions in space such that the flexible implant 30 can travel, or rotate, at or about the flexible section 34 to permit the flexible implant 30 to be delivered and inserted into the desired or selected disc space in a substantially straight approach orientation. In this manner, the flexible implant 30 is manufactured to have the physical properties or characteristics so that it can travel, bend, pivot or rotate about or at one or more reference lines, planes or axes A1, A2 and A3, e.g., as those shown and discussed with respect to FIG. 7. In this manner, the flexible implant 30, via the flexible section 34, can “self balance” or settle into or reach an equilibrium fit or best fit in the interbody disc space after implant 30 insertion. The flexible section 34 enables or permits the implant 30 to seek and find an equilibrium fit or best fit after implant insertion through motion and/or micro motion and flexibility of the flexible section 34 until the flexible implant 30 finds the best anatomic fit in the disc space. This aspect of the flexible implant 30 enhances the biomechanical properties of the implant 30 while the vertebral fusion is setting. Those of skill in the art will recognize that in some embodiments, where required by a patient's anatomy, a physician's requirements or a medical application, the flexible section 34, and by extension the flexible implant 30, can be manufactured so that the implant 30 is only flexible in a selected or desired linear dimensional direction or rotational direction in space during delivery and insertion into the desired or selected disc space in a substantially straight approach orientation.

Those of skill in the art will recognize that the flexible section 34 can be comprised of any bio compatible and flexible material that will permit the implant 30 to bend, deform, pivot or flex about or at the flexible section 34. For example, it may be a deformable plastic, an elastic polymer, an elastomer, rubber or another deformable or elastic material. Further, in one aspect, the flexible implant 32 may be manufactured to have properties or characteristics such that such that the flexible section 34 can or will become rigid or substantially rigid once the implant 30 is fully implanted in the disc space. The flexible section 34 can also be manufactured to become rigid at a desired time or over time after implant insertion. For example, as soon as the implant is inserted in the disc space, over a desired or predetermined time period, or as the fusion is setting. The flexible implant 32 once rigid would thereafter no longer maintain implant flexibility. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

In a lateral approach procedure, the flexible implant 30 arrives at the L5-S1 disc space entry 28 at the angle of approach or insertion angle of approach Z. Prior to implant insertion, the intervertebral disc space is typically prepared with a partial or complete discectomy in order to accept the flexible spinal implant which is to be inserted. In order to minimize damage to the vertebral bodies L5 and S1 and to facilitate entry of the implant into the L5-S1 disc space, it is preferred that the implant enter the L5-S1 disc space in a straight or substantially straight lateral approach orientation. Since the implant 30 arrives at the disc space entry 28 at the angle of approach Z, the implant must bend, deform or deflect such that the implant can enter the L5-S1 disc space in a substantially straight lateral approach orientation. The novel flexible section 34 enables or permits the implant 30 to bend, deform or deflect at or about the flexible section 34 as needed to thereby enable or permit the substantially straight lateral approach entry of the implant 30 into the selected disc space when using a lateral approach procedure.

The ability of the flexible implant to approach and enter the selected disc space in a lateral approach, as opposed to an angled approach results in reduction or minimization of damage or potential damage to the affected vertebral bodies L5 and S1 at the L5-S1 disc space, or to any set disc space vertebral bodies where a flexible implant is delivered and inserted. One advantageous feature of this aspect is that due to the lateral or substantially straight lateral approach of the flexible implant at the disc space, the affected vertebral body endplates be protected against endplate stress and/or fractures that are typically associated with non-flexible or rigid implants which lack the flexibility to transition from a delivery having an angle of approach and reach the disc space and corresponding vertebral bodies and end plates in a lateral or substantially straight lateral orientation. This would in turn reduce or minimize the incidence of post implantation implant subsidence due to endplate stress and/or endplate stress fractures when the endplates are stressed or fractured by a non-flexible or rigid implant which is unable flex and transition from a delivery having an angle of approach to a lateral or substantially straight lateral orientation. The configuration of a non-flexible or rigid implant being delivered at an angle of approach imparts greater implant stresses or forces on a smaller end plate surface area which can result in endplate stress and/or endplate stress fractures which can go undetected until subsequent x-ray imaging reveals the subsidence of the inserted implant. The flexible implant of the present disclosure alleviates or eliminates this drawback of rigid or non-flexible implants.

In this manner, the flexible implant 30 is adapted to bend and turn away from its delivery path orientation, having an insertion angle of approach Z, and enter the disc spaced L5-S1 in a substantially straight lateral approach orientation. As the flexible implant 30 is being delivered, via an instrument attached to the rear attachment section 40 (not shown), the leading end 32 of implant reaches and encounters an obstructing and opposing force at the S1 vertebrae at the disc space entry 28. That opposing force will tend to prevent or retard the entry of the implant into the disc space. This difficulty is overcome in a two fold manner. First, the curved or bullet shaped configuration 38 of the leading end 32 facilitates a smoother entry into the disc space L5-S1 and provides a curved or rounded contour that will facilitate entry and impart distraction of the vertebral bodies L5 and S1 as the implant continues to travel into the disc space. Secondly, the opposing force encountered due to the insertion angle of approach Z is translated through the leading end 32 to the flexible section 34. The flexibility or pliancy of the flexible section 34 permits or enables the implant 30 to bend, deform or deflect as needed about or at the flexible section 34. In this manner, the leading end 32 and the trailing end 36 of the flexible implant 30 will swing or rotate towards a straight lateral orientation that thereby permits the flexible implant 30 to enter the L5-S1 disc space in a substantially straight lateral manner as the implant continues to be inserted or pushed into the disc space L5-S1 by a surgeon. The flexible implant 30, via the flexible section 34, will “self balance” and settle into or reach an equilibrium fit or best fit in the interbody disc space after implant insertion through motion and/or micro motion of the flexible section 34 until the flexible implant 30 settles into the best anatomic fit in the disc space. Once the flexible implant 30 is inserted, the coupled instrument (not shown) can be disconnected from the attachment section 40. In one aspect, the flexible implant 30 will become rigid or substantially rigid once the implant is fully implanted in the disc space at desired time after implant insertion. For example, as soon as the implant 30 is inserted in the disc space, over a desired or predetermined time period, or as the fusion is setting. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

In the depicted lateral approach of FIG. 2, the flexible implant 30 approaches or is delivered at an angle of approach Z measured relative to the straight lateral reference line 20. Those of skill in the art will recognize that the angle of approach Z may be a desired or selected insertion angle of approach depending on the disc space where a flexible implant 30 is to be delivered via a lateral surgical procedure. The insertion angle of approach Z may be different depending on which spinal disc space level the implant is to be delivered at, e.g., the L4-L5 or L5-S1 disc space. Further, those of skill in the art will recognize that the insertion angle of approach may vary to accommodate a patient's or physician's needs and requirements during surgery. In one aspect, the desired insertion angle of approach is in the range of between five to forty-five degrees (5°-45°), with a preferred range of between ten and thirty degrees (10°-30°).

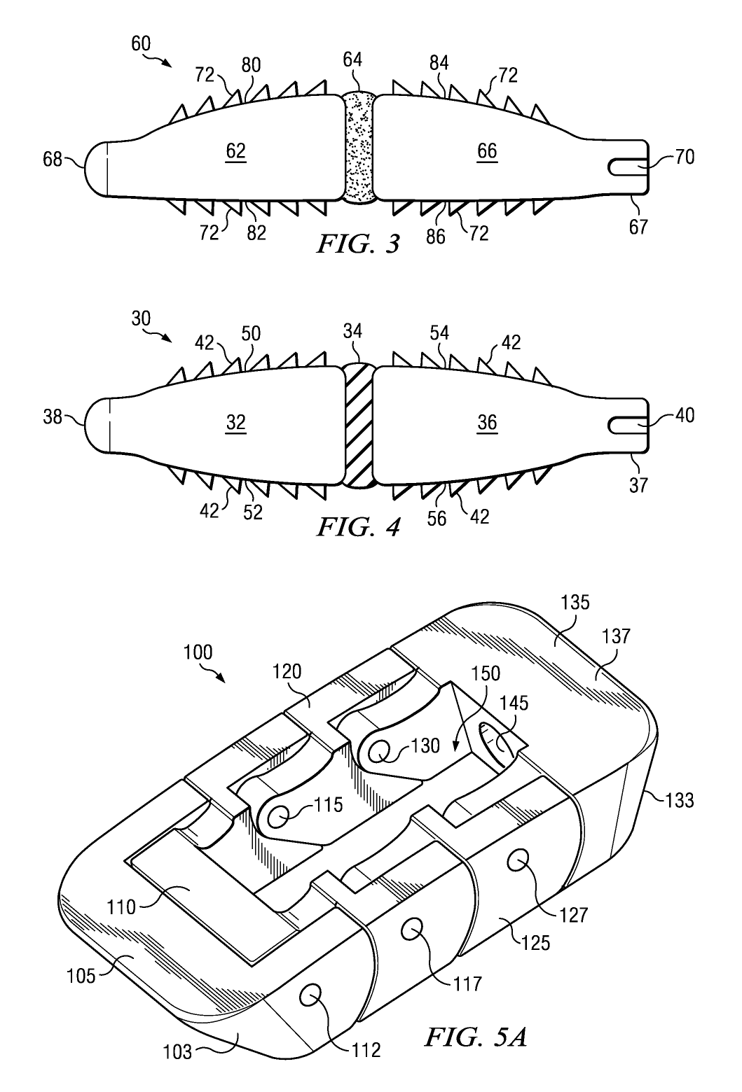

FIG. 3 shows a side view of a flexible spinal implant 60 according to another aspect of the present disclosure having a physical configuration adapted for implant 60 insertion at the L5-S1 disc space. The flexible spinal implant 60 comprises a leading end 62, a trailing end 66 and a flexible section 64 connecting the leading end 62 and the trailing end 32. The leading end 62 and the trailing end 66 respectively comprise curved or convex upper walls 80 and 84 to compliment the curved or concave nature of the L5 vertebral body end plate 15 when the implant is in place in the L5-S1 disc space. The leading end 62 and the trailing end 66 respectively comprise substantially flat lower walls 82 and 86 intended to compliment the relatively flat nature of the S1 sacral vertebrae when the implant is in place in the L5-S1 disc space. Those of skill in the art will recognize that other surface configurations, e.g., circular, oval, angled, etc., may be used instead depending on patient anatomy and physician requirements.

The flexible implant 60 further also comprises anti-back out protrusions 72 on the upper and lower surfaces 80, 82, 84 and 86, and an instrument attachment section 70. The anti-back out protrusions 72 extending from the upper and lower surfaces 80, 82, 84 and 86 will be configured and oriented so as to prevent the implant 60 from backing out or being ejected after insertion into the disc space. In the aspect shown in FIG. 3, the anti-back out protrusions 72 have a triangular configuration and are oriented back toward the trailing proximal implant end 67 of the implant 60.

The leading end 62 has a physical configurations adapted to facilitate insertion into the disc space L5-S1. In one aspect, the leading end 62 has a curved or bullet shaped surface 68 which facilitates insertion of the flexible implant 60 in the L5-S1 disc space. The curved or bullet shaped nose 68 will impart a distracting force between the L5-S1 disc space to facilitate insertion of the flexible implant 60. The trailing end 66 comprise an implant grip or attachment section 70 situated at the proximal implant end 77 which enables the coupling of an insertion instrument (not shown). The attachment section 70 enables for the controlled delivery of the flexible implant 70 into the L5-S1 disc space via a lateral approach. The attachment section 70 is preferably a recessed into the trailing end 66 such that when an instrument is coupled to the flexible implant 60, the instrument is entirely interior to the exterior surface of the proximal implant end 67. In one aspect, the attachment section is a recessed slot 70 on both sides of the proximal implant end 67.

The flexible section 64 preferably connects the leading end 62 and the trailing end 66 to form the flexible spinal implant 60. The flexible section 64 is preferably located at a mid portion of the flexible spinal implant 60. However, those of skill in the art will recognize that the flexible section 64 may be located at other positions of the flexible spinal implant 60 as may be desired or required by a physician or patient anatomy, or the needs or requirement of a medical procedure. The flexible section 64 is coupled or attached between the leading end 62 and the trailing end 66 so as to form a single assembled flexible spinal implant 60. The flexible section 64 permits or enables the implant to bend, flex or pivot at or about the flexible section 64 when the implant is being inserted into the L5-S1 disc space via a preferred later surgical approach.

The flexible section 64 also permits the implant 60 to be fully flexible in any one or more dimensional directions in space such that the flexible implant 60 can travel or rotate at or about the flexible section 64 to permit the implant 60 to be delivered and inserted into the desired or selected disc space in a substantially straight approach orientation. In this manner, the flexible implant 60 is manufactured to have the physical properties or characteristics so that it can travel, bend, pivot or rotate about or at one or more reference lines, planes or axes A1, A2 and A3, e.g., as those shown and discussed with respect to FIG. 7. In this manner, the flexible implant 60, via the flexible section 64, can “self balance” or settle into or reach an equilibrium fit or best fit in the interbody disc space after implant insertion. The flexible section 64 permits the implant 60 to seek and find a fit or equilibrium fit after implant insertion through motion and/or micro motion and flexibility of the flexible section 64 until the flexible implant 60 finds the best anatomic fit in the disc space. This aspect of the flexible implant 60 enhances the biomechanical properties of the implant 60 while the vertebral fusion is setting. Those of skill in the art will recognize that in some embodiments, where required by a patient's anatomy or a physician's requirements, the flexible section 64, and by extension the flexible implant 60, can be manufactured so that the implant 60 is only flexible in a selected or desired linear dimensional direction or rotational direction in space during delivery and insertion into the desired or selected disc space in a substantially straight approach orientation.

The flexible section 64 can be comprised of a biocompatible and flexible material that will permit the implant to bend or flex about or at the mid section 64. For example, a deformable plastic, an elastic polymer, an elastomer, rubber or another elastic material. In one aspect, the flexible implant 60 may be manufactured to have properties or characteristics such that such that the flexible section 64 can or will become rigid or substantially rigid once the implant is fully implanted in the disc space. The flexible implant section 64 can be manufactured to become rigid at a desired time or over time after implant insertion. For example, as soon as the implant is inserted in the disc space, over a desired or predetermined time period, or as the fusion is setting. In one aspect, the implant rigidity characteristic may be provided through the use of shape memory nitinol or other shape memory materials which can reach rigidity in a patient anatomical environment. The flexible implant 60 once rigid would thereafter no longer maintain implant flexibility. This aspect or property may be used where desired or required by a patient's anatomy or a physician's requirement.

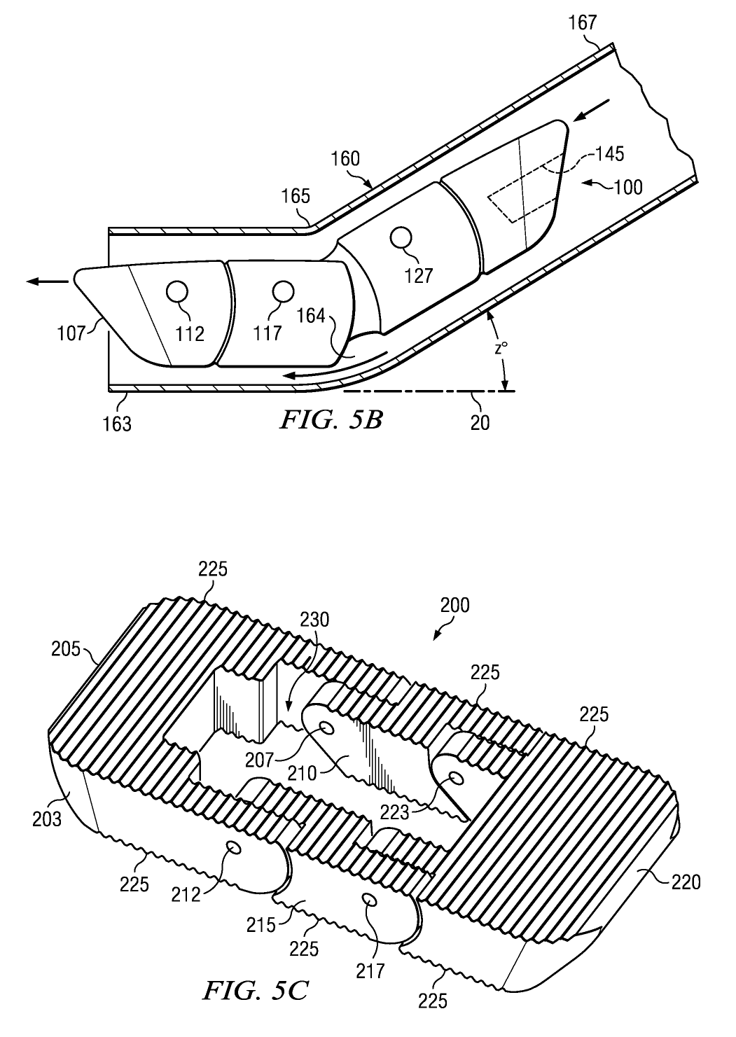

FIG. 5A shows an isometric view of a flexible spinal implant 100 according to another aspect of the present disclosure. FIG. 5B shows a side view of the flexible spinal implant 100 of FIG. 5A in an implant insertion channel 160 that can be positioned for implant insertion at a selected disc space, e.g., L4-L5 or L5-S1 shown in FIG. 1, via a lateral approach procedure. The flexible spinal implant 100 is a multi-component pivoting assembly comprising a leading end 105, a first member 110, a second member 120, a third member 125 and a trailing end 135.

The leading end 105 is pivotally connected to the first member 110 at a first hinge 112 to thereby permit rotational motion between the leading end 105 relative to the first member 110. The first member 110 is pivotally connected to the second member 120 at a second hinge 115 to thereby permit rotational motion of the first member 110 relative to the second member 120. The first member 110 is pivotally connected to the third member 125 at a third hinge 117 to thereby permit rotational motion of the first member 110 relative to the third member 125. The trailing end 135 is pivotally connected to the second member 120 at a fourth hinge 130 to thereby permit rotational motion of the trailing end 135 relative to the second member 120. The trailing end 135 is pivotally connected to the third member 125 at a fifth hinge 127 to thereby permit rotational motion of the trailing end 110 relative to the third member 125.

As shown in FIGS. 5A and 5B, the leading end 105 has a physical configuration adapted to facilitate or ease insertion of the flexible implant 100 into a disc space. In a preferred aspect, shown in FIGS. 5A and 5B, the leading end 105 has a wedge type shape 103 which facilitates insertion of the flexible implant 100 into a disc space. The wedge shaped nose 103 of the leading end 105 may, if the disc space is collapsed, impart a distracting force to adjacent collapsed vertebrae as the flexible implant 100 travels or is inserted into the disc space.

The trailing end 135 comprises an implant grip or attachment aperture 145 situated at the proximal implant end 137 which enables the coupling of an instrument (not shown) to the flexible spinal implant 100. The attachment aperture 145 enables an instrument to couple to the flexile spinal implant 100 for delivery of the flexible implant 100 through an implant insertion channel 160 into a selected disc space via a lateral approach. After insertion of the flexible implant 100, the attachment aperture 145 can also be used to insert graft material, as discussed previously, if none was packed in prior to implant insertion.

The flexible spinal implant 100 further comprises an interior implant aperture 150 defined and formed by the pivotally connected first member 110, second member 120, third member 125 and trailing end 135. The interior implant aperture 150 can be filled or packed with graft material before or after insertion of the flexible implant 100 into the selected disc space. The graft material may be composed of material that has the ability to promote, enhance and/or accelerate bone growth and fusion of vertebral bodies. Graft material may include allograft material, bone graft, bone marrow, demineralized bone matrix putty or gel and/or any combination thereof. The filler graft material may promote bone growth through and around the interior implant aperture 150 to promote fusion of the disc space intervertebral joint. Those of skill in the art will recognize that the use of filler graft material is optional, and it may or may not be used depending on the needs or requirements of a physician or a medical procedure.

The first member 110, second member 120 and third member 125 are pivotally connected to each other and to the leading end 105 and trailing end 135 to form the multi-piece flexible implant 100 shown in FIGS. 5A and 5B. The pivoting connections 112, 115, 117, 127 and 130 permit or enable the flexible spinal implant 100 to pivot or articulate about the pivoting connections 112, 115, 117, 127 and 130 such that the flexible spinal implant 100 can bend and articulate as may be needed to permit delivery and insertion of the flexible implant 100 in a lateral approach. For example, implant insertion into disc space L4-L5 or L5-S1 shown in FIGS. 1 and 2. Those of skill in the art will recognize that the flexible implant 100 can have a different number of implant components and corresponding pivoting connections, for example, as shown in FIG. 5C which comprises two pairs of pivoting connections 207, 212, 217 and 223. The number of pivoting connections will depend on the angle of approach Z that the flexible implant 100 will be inserted at, or an insertion channel bend or turn 165 that the flexible implant 100 will traverse as the flexible implant 100 travels through the insertion channel 160. In one aspect, the greater the approach angle Z, the larger the number of implant components and corresponding pivoting connections required to enable the implant to sufficiently articulate in order to traverse the insertion channel bend 165.

In one aspect, the flexible implant 100 may be manufactured to have properties or characteristics such that the pivoting connections 112, 115, 117, 127 and 130 can or will become rigid or substantially rigid once the implant 100 is fully implanted in the disc space. The pivoting connections 112, 115, 117, 127 and 130 can be manufactured to become rigid at a desired time or over time after implant insertion. For example, as soon as the implant is inserted in the disc space, over a desired or predetermined time period, or as the fusion is setting. The pivoting connections 112, 115, 117, 127 and 130, once rigid, would thereafter no longer permit the implant 100 to maintain implant flexibility. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

The flexible implant 100 of FIG. 5A is preferably delivered to a disc space via an adjacently positioned implant insertion channel 160 in a lateral approach procedure. The following contemplates a delivery at the L4-L5 or L5-S1 disc space. However, those of skill in the art will recognize that the insertion described below may be carried out at other spinal disc levels. An implant insertion channel 160 is positioned adjacent the disc space L4-L5 or L5-S1 where the flexible implant 100 is to be inserted. The implant insertion channel 160 comprises a distal first channel end 163, a channel turn section 164 and a proximal second channel end 167. The implant insertion channel 160 is preferably a channel with a cross section that compliments the exterior physical configuration of the flexible implant 100 that will travel inside the insertion channel 160. In a preferred aspect, the implant insertion channel 160 has a rectangular cross-section. However, other configurations may be used, as appropriate, to compliment the flexible implant 100, e.g., circular, squared, etc.

The implant insertion channel 160 transitions from the first channel end 163 to the channel turn section 164 and then to the second channel end 167, as shown in FIG. 5B. The channel turn section 164 is oriented such that the first channel end 163 and the second channel end 167 describe an angle of approach Z. The channel turn section 164 is typically fixed. However, those of skill in the art will recognize that the channel turn section 164 could also be adjustable. For example, through a hinged arrangement between the first channel end 163 and the second channel end 167. In this manner, the implant insertion channel 160 could be adjusted to define a variety or range of angles of approach Z measured relative to the straight lateral reference line 20. The angle of approach Z may differ depending on the disc space where a flexible implant 100 is to be delivered and inserted via a lateral surgical procedure. The insertion angle of approach Z may also vary to accommodate a patient's or physician's needs and requirements during surgery. In one aspect, the desired insertion angle of approach Z is between five to forty-five degrees (5°-45°), with a preferred range of between ten and thirty degrees (10°-30°).

In a preferred aspect, the flexible implant 100 travels inside the implant insertion channel 160, as shown in FIG. 5B, to reach the desired or selected disc space level. In order for the flexible implant 100 to reach and enter the disc space in a substantially straight lateral approach orientation, the traveling flexible implant 100 will be guided by the interior walls of the implant insertion channel 160. The channel turn section 164 interacts with and forces the flexible implant 100 to actuate and pivot about the pivoting connections 112, 115, 117, 127 and 130 as the flexible implant 100 travels through the channel turn section 164. This interaction imparts a force to the flexible spinal implant 100 such that the flexible spinal implant 100 articulates to thereby enable travel through the channel turn section 164. In this manner, the flexible implant 100 is adapted to articulate and turn from its delivery path having an insertion angle of approach Z and enter the disc space in a substantially straight lateral approach orientation. The forced articulation by the channel turn section 164 interaction, in particular, transitions the flexible implant 100 from an angled lateral approach Z at the second channel end 167 to a substantially straight lateral approach orientation in the first channel end 163, as shown in FIG. 5B. As the flexible implant 100 continues to travel inside the implant insertion channel 160, the flexible implant 100 will enter the selected disc space in a substantially lateral approach orientation. The flexible implant 100, via the pivoting connections 112, 115, 117, 127 and 130, will “self balance” and settle into or reach an equilibrium fit or best fit in the interbody disc space after implant insertion through motion and/or micro motion of the pivoting connections 112, 115, 117, 127 and 130 until the flexible implant 1000 comes to the best anatomic fit in the disc space. The flexible implant 100 can be delivered via an instrument (not shown) coupled to the attachment aperture 145 via travel through the implant insertion channel 160 into the selected disc space. Once the flexible implant 100 is inserted in the disc space, the coupled instrument can be disconnected from the attachment aperture 145.

FIG. 5C shows an isometric view of a flexible spinal implant 200 according to another embodiment of the present disclosure that can be inserted at a selected disc space level, e.g., L4-L5 or L5-S1, via a lateral approach procedure. The flexible spinal implant 200 is a multi-component implant pivoting assembly comprising a leading end 205, a first member 210, a second member 215 and a trailing end 220. The leading end 205 is pivotally connected to the first member 210 at a first hinge 207 to thereby permit rotational motion between the leading end 205 relative to the first member 210. The leading end 205 is pivotally connected to the second member 215 at a second hinge 212 to thereby permit rotational motion between the leading end 205 relative to the second member 215. The trailing end 220 is pivotally connected to the first member 210 at a third hinge 223 to thereby permit rotational motion of the trailing end 220 relative to the first member 210. The trailing end 220 is pivotally connected to the second member 215 at a fourth hinge 217 to thereby permit rotational motion of the trailing end 220 relative to the second member 215.

The leading end 205 has a physical configuration adapted to facilitate or ease insertion of the flexible implant 200 into a disc space. In a preferred aspect, shown in FIG. 5C, the leading end 205 has a wedge type contour 203 which facilitates insertion of the flexible implant 200 into a disc space. The wedge shaped nose 203 of the leading end 205 may serve to impart a distracting force to adjacent vertebrae as the flexible implant 200 travels or is inserted into a disc space.

The flexible spinal implant 200 further comprises an interior implant aperture 230 defined by the pivotally connected leading end 205, first member 210, second member 215 and trailing end 220. The interior implant aperture 230 can be filled with a graft material before insertion of the flexible implant 200 into a selected disc space. The graft material may be composed of material that has the ability to promote, enhance and/or accelerate bone growth and fusion of vertebral bodies. The graft material may promote bone growth through and around the interior implant aperture 230 to promote fusion of the disc space intervertebral joint. The use of filler graft material is optional, and it may or may not be used depending on the needs or requirements of a physician or a medical procedure.

The flexible implant 200 also comprises anti-back out protrusions 225 on the upper and lower surfaces of the flexible implant 200. The anti-back out protrusions 225 extending from the upper and lower surfaces are preferably configured and oriented so as to prevent the implant 200 from backing out or being ejected after insertion into a disc space. In the aspect shown in FIG. 5C, the anti-back out protrusions 225 have a triangular ridge configuration that traverse across the upper and lower surfaces of the leading end 205, the first member 210, the second member 215 and the trailing end 220 of the flexible implant 200. Those of skill in the art will recognize that the protrusions can have other shapes, configurations or sizes including, among others, pyramids, triangles, cones, spikes and keels.

The first member 210 and second member 215 are pivotally connected to each other and to the leading end 205 and trailing end 220 to form the multi-component flexible implant 200 shown in FIG. 5C. The pivoting connections 207, 212, 217 and 223 permit or enable the flexible spinal implant 200 to pivot or articulate about the pivoting connections 207, 212, 217 and 223 such that the flexible spinal implant 200 can bend and articulate as may be needed to permit delivery and insertion of the flexible implant 200 in a disc space via a lateral approach. For example, into disc space L4-L5 or L5-S1 shown in FIGS. 1 and 2. As discussed previously, the flexible implant 200 can have a different number of implant components and corresponding pivoting connections. The number of pivoting connections will depend on the angle of approach Z that the flexible implant 200 will be inserted at. In one aspect, the flexible implant 200 may be manufactured to have properties or characteristics such that such that the pivoting connections 207, 212, 217 and 223 can or will become rigid or substantially rigid once the implant 200 is fully implanted in the disc space. The pivoting connections 207, 212, 217 and 223 can be manufactured to become rigid at a desired time or over time after implant insertion. For example, as soon as the implant is inserted in the disc space, over a desired or predetermined time period, or as the fusion is setting. The pivoting connections 207, 212, 217 and 223 once rigid would thereafter no longer maintain implant flexibility. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

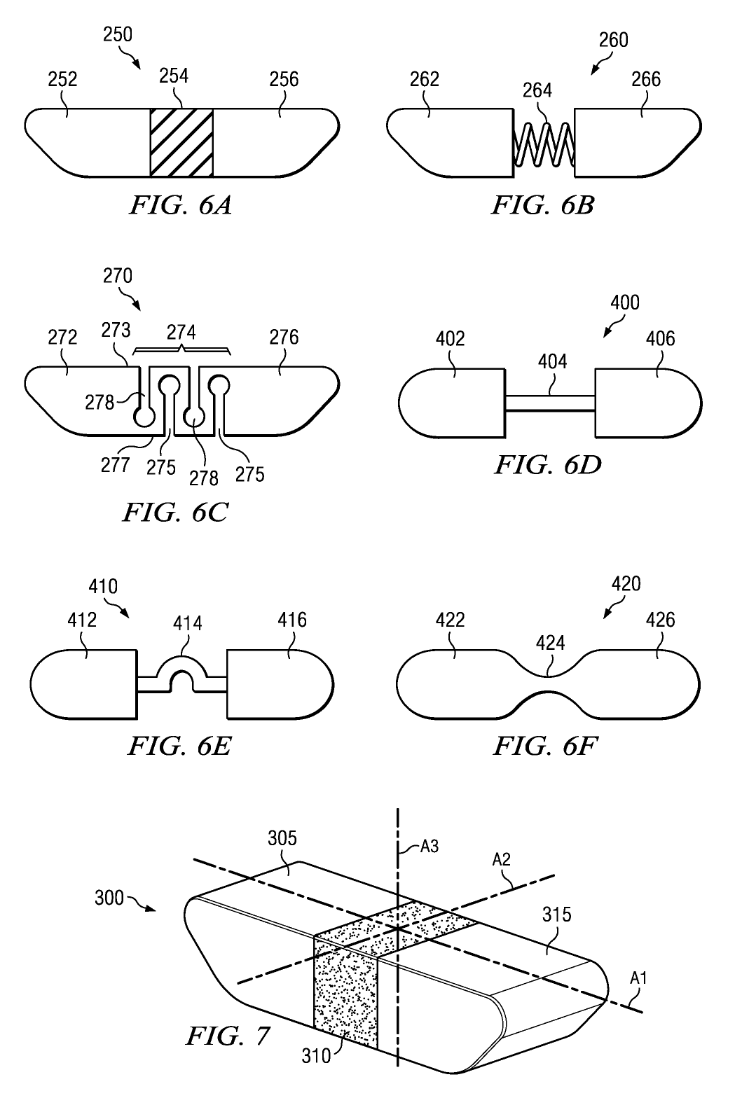

FIGS. 6A-6E show side views of flexible spinal implants 250, 260, 270, 400 and 410 which disclose other flexible section aspects 254, 264, 274, 404 and 410 contemplated in the present disclosure. The flexible sections 254, 264, 274, 404 and 410 enable the respective flexible implants 250, 260, 270, 400 and 410 to bend, flex, rotate or pivot at or about the flexible section 254, 264, 274, 404 and 410 so that an implant can enter the disc space in a substantially lateral approach orientation. The flexible implant 250, 260, 270, 400 and 410, via the flexible section 254, 264, 274, 404 and 410, will “self balance” and settle into or reach an equilibrium fit or best fit in the interbody disc space after implant insertion through motion and/or micro motion of the flexible section 254, 264, 274, 404 and 410 until the flexible implant 250, 260, 270, 400 and 410 comes to the equilibrium or best anatomic fit in the disc space. The flexible implants 250, 260, 270, 400 and 410, shown in FIGS. 6A-6E, are preferably used for delivery and insertion in a disc space, such as L4-L5 or L5-S1, in a lateral approach procedure. However, those of skill in the art will recognize that they may be delivered and inserted at other spinal disc levels.

FIG. 6A shows a flexible spinal implant 250 comprising a leading end 252, a trailing end 256 and a flexible section 254 connecting the leading end 252 and the trailing end 256. The flexible section 254 can be comprised of any bio compatible and flexible material that permits the implant to bend, rotate or flex about or at the flexible section 254, including a deformable plastic, an elastic polymer, an elastomer, rubber or another elastic material. FIG. 6B shows a flexible spinal implant 260 comprising a leading end 262, a trailing end 266 and a flexible section 264 connecting the leading end 262 and the trailing end 266. The flexible section 264 shown in FIG. 6B is contemplated as a flexible metallic section that is bio compatible and made of resilient flexible metallic material that permits the implant to bend, rotate or flex about or at the flexible section 264. In the aspect, shown in FIG. 6B, the flexible section 264 is preferably a bio compatible spring type mechanism which permits the implant 260 to bend, rotate or flex about or at the mid section 264.

FIG. 6C shows a flexible spinal implant 270 comprising a leading end 272, a trailing end 276 and a flexible section 274 connecting the leading end 272 and the trailing end 276. The flexible section 274 is contemplated as a series of slots 275 and 278 formed in the implant body to form the flexible section 274. The implant slots 275 and 278 form the flexible section 274 that permit the implant 270 to bend or flex about or at the flexible section 274. In another aspect, the flexible section 274 may be comprised of a different number of slots 275 and 278. In another aspect, the flexible section 274 may be comprised of slots 275 that are formed in the lower implant surface 277 or slots 278 that are formed in the upper implant surface 273.

FIGS. 6D and 6E show a flexible spinal implant 400 and 410 comprising a leading end 402 and 412, a trailing end 406 and 416 and a flexible section 404 and 414 connecting the leading end 402 and 412 and the trailing end 406 and 416, respectively. The flexible section 404 and 414 shown in the aspects of FIGS. 6D and 6E are a flexible metallic section that is bio compatible and made of resilient flexible metallic material that permits the implant 400 and 410 to bend, rotate or flex about or at the flexible section 404 and 414. In the aspect shown in FIG. 6D, the flexible section 404 is a flat metallic section or a leaf spring mechanism that is bio compatible and which permits the implant 400 to bend or flex about or at the flexible section 404. In the aspect shown in FIG. 6E, the flexible section 414 is a curved metallic section that is bio compatible and which permits the implant 410 to bend, rotate or flex about or at the flexible section 414. The flexible section 404 and 414 can be comprised of a biocompatible metallic material such as, among others, stainless steel, titanium, nitinol, platinum, tungsten, silver, palladium, cobalt chrome alloys, shape memory nitinol and mixtures thereof. The biocompatible metallic material used can depend on the patient's need and physician requirements.

FIG. 6F shows a flexible spinal implant 420 comprising a leading end 422, a trailing end 426 and a flexible section 424 connecting the leading end 422 and the trailing end 426. The flexible section 424 is contemplated as a portion of the implant which is formed as part of the implant body 420 having a reduced size or being a thin implant section in the implant body 420. The reduced size section or thin implant section forms the flexible section 424 which is manufactured to permit the implant 420 to bend, rotate or flex about or at the flexible section 424.

In a further aspect contemplated for the flexible implants 250, 260, 270, 400, 410 and 420, shown in FIGS. 6A-6F, the flexible implant 250, 260, 270, 400, 410 and 420 may be manufactured to have properties or characteristics such that such that the flexible section 254, 264, 270, 404, 414 and 424 can or will become rigid or substantially rigid once the implant is fully implanted in the disc space. The flexible implant section 254, 264, 270, 404, 414 and 424 can be manufactured to become rigid at a desired time or over time after implant insertion. For example, as soon as the implant is inserted in the disc space, over a desired or predetermined time period, or as the fusion is setting. The flexible implant 250, 260, 270, 400, 410 and 420, once rigid, would thereafter no longer maintain implant flexibility. In one aspect, the implant rigidity characteristic may be provided through the use of shape memory nitinol or other shape memory materials which can reach rigidity in a patient anatomical environment. This aspect or property may be used where desired or required by a patient's anatomy or a physician's requirement.

FIG. 7 shows an isometric view of a flexible spinal implant 300 according to a further aspect the present disclosure. Similar to flexible implant aspects already discussed, the flexible section 310 enables or permits the flexible implant 300 to bend, rotate, flex or pivot at or about the flexible section 310 so that an implant can enter the disc space in a substantially straight approach orientation, e.g., a straight lateral approach orientation. The flexible implant 300 may be delivered to a selected disc space such as L4-L5 or L5-S1, as discussed herein, or other desired spinal disc levels. The flexible spinal implant 300 can be delivered and inserted into a desired disc space via a spinal surgical approach and procedure selected or required by a physician.

The flexible spinal implant 300 can be delivered and inserted into a desired disc space via a lateral approach procedure, such as a DLIF procedure, to clear the obstructing iliac crest, and neurological and vascular structures. The flexible spinal implant 300 may also be delivered and inserted into a desired disc space via a PLIF or TLIF procedure to bend around and safely bypass or clear the cauda equina. In one aspect, the flexible spinal implant 300 is delivered via or through a minimal access spinal technology (MAST) surgical technique or procedure. Those of skill in the art will recognize that the flexible spinal implant 300 may also be delivered and inserted via other known surgical approaches, including, a posterior, direct lateral, translateral, posterolateral, or anterolateral or any suitable oblique direction. Some known techniques and approaches that may be used to insert the flexible implant 300 may also include, among others, anterior lumbar interbody fusion (ALIF). Further, those of skill in the art will recognize that a spinal implant may be delivered and inserted through known surgical technique and procedures, including: open, mini-open or other minimally invasive surgical (MIS) techniques.

FIG. 7 shows a flexible spinal implant 300 comprising a leading end 305, a trailing end 315 and a flexible section 310 connecting the leading end 305 and the trailing end 315. FIG. 7 additionally shows three dimensional (3D) implant reference lines, planes or axes A1, A2 and A3. The 3D implant reference lines, planes or axes A1, A2 and A3 may be a selected or desired reference line, plane or axis. Those of skill in the art will recognize that the 3D implant reference lines, planes or axes A1, A2 and A3 may also, or instead, be known references lines, planes or axes such as the traditional x-y-z axes, or lines, planes or axes that represent Axial, Sagittal or Coronal anatomical planes. The flexible section 310 can be comprised of any bio compatible and flexible material that permits the implant to bend or flex about or at the flexible section 310, including a deformable plastic, an elastic polymer, an elastomer, rubber or another elastic material.

The flexible section 310, shown in FIG. 7, further illustrates another advantageous aspect of the flexible implant 300. The flexible section 310 permits the implant to travel, bend, rotate, pivot or flex about or at the flexible section 310, in or along any one of the 3D implant reference lines, planes or axes A1, A2 and A3 as the implant 300 is being delivered and inserted into the desired or selected disc space, or as the implant 300 is swinging, bending or turning away from its delivery path orientation to thereby enter the disc space in a substantially straight approach orientation. The flexible section 310 also permits the implant 300 to travel, bend, rotate, pivot or flex about or at the flexible section 310, in any three dimensional direction or orientation with respect to the 3D implant reference lines, planes or axes A1, A2 and A3 as the implant 300 is being delivered and inserted into the desired or selected disc space, or as the implant is swinging, bending, rotating or turning away from its delivery path orientation to thereby enter the disc space in a substantially straight approach orientation. The flexible section 310 further permits the implant 300 to rotate, travel, bend, rotate, pivot or flex about or at the flexible section 310, in any one or more dimensional direction or orientation with respect to the 3D implant reference lines, planes or axes A1, A2 and A3 as the implant 300 is being delivered and inserted into the desired or selected disc space, or as the implant 300 is swinging, bending, rotating or turning away from its delivery path orientation to thereby enter the disc space in a substantially straight approach orientation.

The flexible section 310 thereby permits the implant 300 to be fully flexible, deformable or moveable in any one or more dimensional directions in space such that the flexible implant 300 can travel, bend, pivot or rotate at or about the flexible section 310 to permit the flexible implant 300 to be delivered and inserted into the desired or selected disc space in a substantially straight approach orientation. In this manner, the flexible implant 300 has the physical properties or characteristics so that it can travel, bend, pivot or rotate about or at one or more reference lines, planes or axes A1, A2 and A3. In this manner, the flexible implant 300, via the flexible section 310 can “self balance” or settle into or reach an equilibrium fit or best fit in the interbody disc space after implant 300 insertion. The flexible section 310 permits the implant 300 to reach a fit or equilibrium fit after implant insertion through motion and/or micro motion and flexibility of the flexible section 310 until the flexible implant 300 settles into the best anatomic fit in the disc space. This aspect of the flexible implant 300 enhances the biomechanical properties of the implant 300 while the vertebral fusion is setting. This novel aspect discussed with respect to FIG. 7 is also contemplated for the flexible implants previously discussed in relation to FIGS. 1-6C. Those of skill in the art will recognize that in some embodiments, where required by a patient's anatomy, a surgical procedure or a physician's requirements, the flexible section 310, and by extension the flexible implant 300, could be manufactured so that the implant 300 is only flexible in a selected or desired linear dimensional direction or rotational direction in space during delivery and insertion into a disc space in a substantially straight approach orientation.

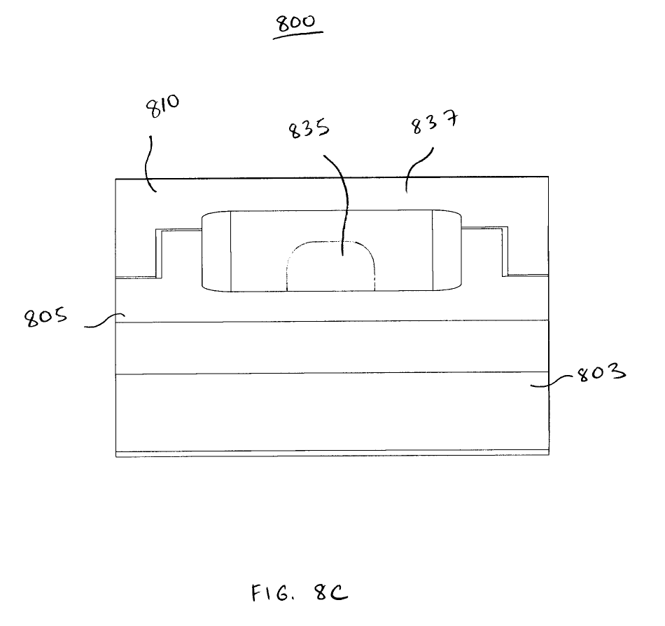

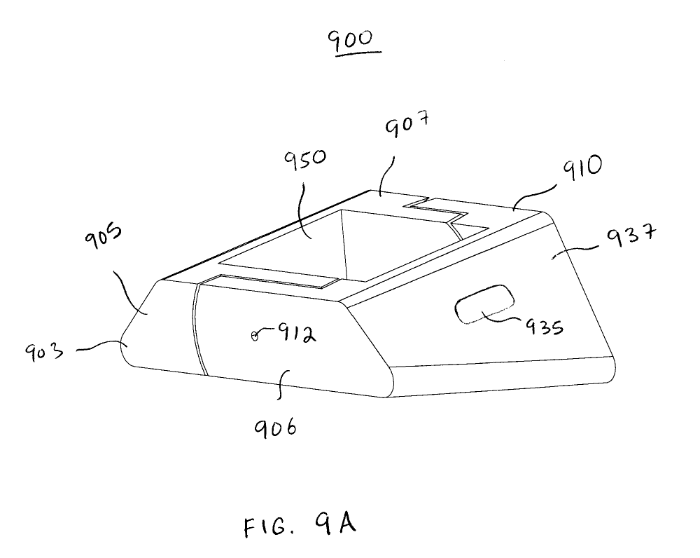

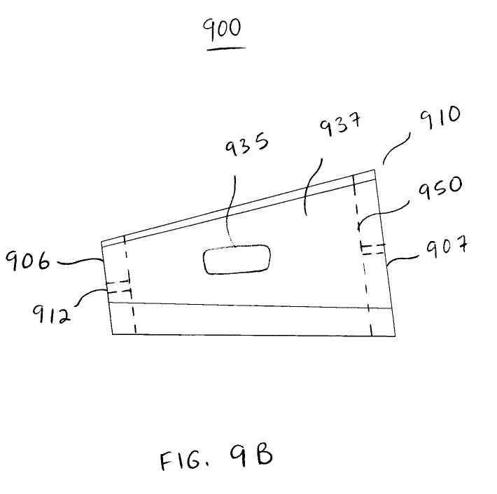

FIG. 8A shows an isometric view of a flexible spinal implant 800 according to another embodiment of the present disclosure. FIGS. 8B and 8C show side and front views of the flexible implant 800 of FIG. 8A. The flexible implant 800 comprises substantially a wedge shape which enables the flexible implant 800 to correct or alleviate coronal spinal deformities by imparting the angled configuration of the implant wedge shape to the adjacent vertebral member in a coronal orientation when the implant is inserted via a lateral approach and positioned between adjacent vertebral bodies in a selected intervertebral disc space. In this manner, the angled configuration of the wedge shape of the inserted implant 800 can be imparted to the adjacent vertebral bodies to thereby correct, improve or stabilize the coronal spacing, curvature, orientation or alignment of the selected disc space and overall spinal anatomy. The flexible implant 800 can be delivered and inserted at a selected disc space, e.g., L4-L5 or L5-S1 shown in FIG. 1, via a lateral approach procedure, as described previously in relation to FIG. 5B or 5C. Such a delivery and insertion may be accomplished via an appropriate sized and configured implant insertion channel (not shown) which would be positioned adjacent the selected disc space. Those of skill in the art will recognize that in other aspects, the flexible implant 800 could also be delivered and inserted into the disc space via other approaches which would then correct or alleviate spinal deformities in another anatomical plane or combination of anatomical planes by imparting the angled configuration of the implant wedge shape to the adjacent vertebral member in that orientation when the implant is inserted and positioned between adjacent vertebral bodies in a selected intervertebral disc space. For example, if the flexible implant 800 is delivered and inserted into the disc space via a posterior approach, the implant would correct or alleviate sagittal spinal deformities by imparting the angled configuration of the implant wedge shape to the adjacent vertebral member in a sagittal orientation when the implant 800 is inserted and positioned between adjacent vertebral bodies in a selected intervertebral disc space.

The flexible implant 800 of FIGS. 8A-8C comprises a multi-component flexible assembly comprising a leading end 805 pivotally connected to a trailing end 810. The flexible implant 800 comprises substantially a wedge shape or configuration. The implant 800 has an overall wedge shaped configuration where the implant's leading end 805 is the narrow part of the wedge and the implant's opposite trailing end 810 is the wider or taller part of the wedge configuration. Inserting the wedge shaped flexible implant 800 in a selected disc space permits or enables a physician to impart the angled configuration of the implant's wedge shape to the adjacent vertebral bodies to thereby correct, improve or stabilize the coronal spacing, curvature, orientation or alignment of the selected disc space and in turn the overall coronal spinal anatomy.

The flexible implant assembly has a flexible section comprised of the leading end 805 pivotally connected to the trailing end 810 at a first hinge 812 to thereby permit rotational motion between the leading end 805 relative to the trailing end 810. The leading end 805 has a physical configuration adapted to facilitate or ease insertion of the flexible implant 800 into a disc space. In this aspect, the leading end 805 has a wedge shape nose 803. The wedge shaped nose 803 can impart a distracting force to adjacent vertebrae as the flexible implant 800 travels or is inserted into the disc space. The leading end 805 is pivotally connected to the trailing end 810 to form a hinged multi-piece flexible implant 800 shown in FIGS. 8A-8C. The flexible section is a pivoting connection 812 that permits or enables the flexible spinal implant 800 to pivot or articulate about the pivoting connection 812 such that the flexible spinal implant 800 can bend and articulate as may be needed to permit delivery and insertion of the flexible implant 800 in a lateral approach, including to enable the implant to sufficiently articulate in order to traverse an insertion channel bend.

The flexible spinal implant 800 further comprises an interior implant aperture 850 defined and formed by the pivotally connected leading end 805 and trailing end 810. The interior implant aperture 850 can be filled or packed with graft material before or after insertion of the flexible implant 800 into the selected disc space. The trailing end 810 also comprises an implant grip or attachment aperture 835 situated at the proximal implant end 837 which enables the coupling of an instrument (not shown) to the flexible spinal implant 800. The attachment aperture 835 would enable an instrument to couple to the flexible spinal implant 800 and deliver the flexible implant 800 through an implant insertion channel (not shown) into a selected disc space via a lateral approach.

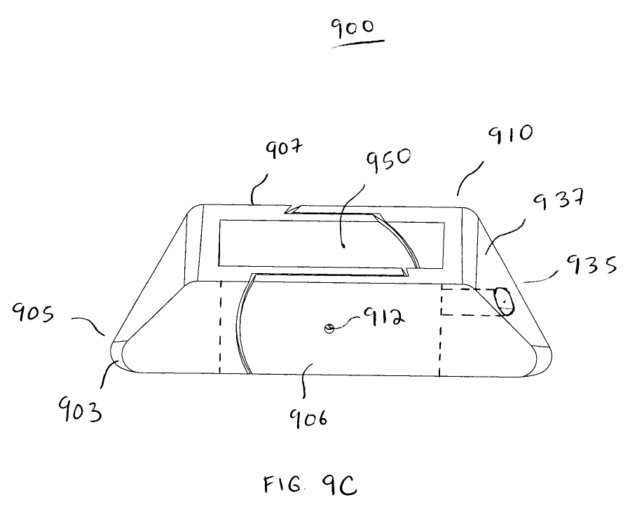

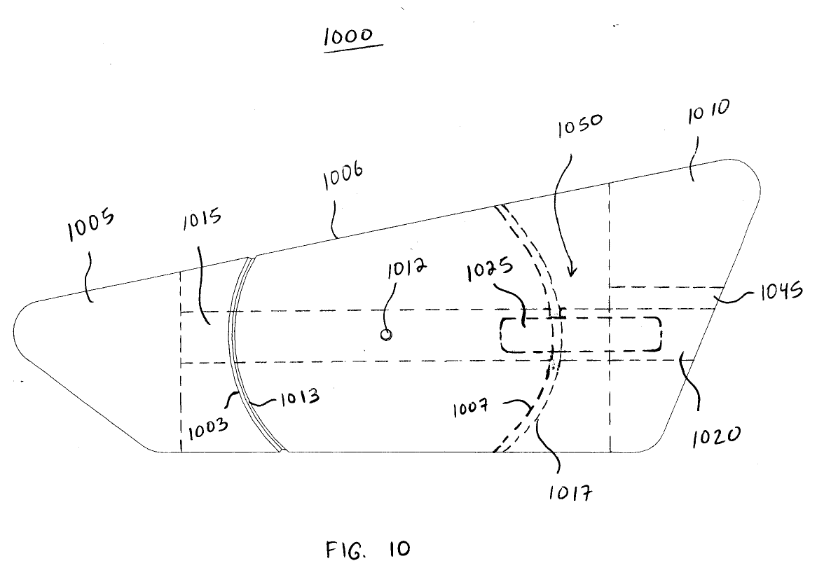

FIG. 9A shows an isometric view of a flexible spinal implant 900 according to another embodiment of the present disclosure. FIGS. 9B and 9C show side and front views of the flexible implant 900 of FIG. 9A. The flexible implant 900 comprises substantially a wedge shape configuration in a lateral or side-to-side direction and orientation of the implant which enables the flexible implant 900 to correct or alleviate sagittal spinal deformities by imparting the side-to-side or lateral angled configuration of the implant wedge shape to the adjacent vertebral member in a sagittal orientation when the implant is inserted via a lateral approach and positioned between adjacent vertebral bodies at a selected intervertebral disc space. In this manner, the lateral angled configuration of the wedge shape of the inserted implant 900 can be imparted to the adjacent vertebral bodies to thereby correct, improve or stabilize the sagittal spacing, curvature, orientation or alignment of the selected disc space and overall spinal anatomy. The flexible implant 900 can also be delivered and inserted at a selected disc space via a lateral approach procedure, as described previously in relation to FIG. 5B or 5C. Such a delivery and insertion may be accomplished via an appropriate sized and configured implant insertion channel (not shown) which would be positioned adjacent the selected disc space. Those of skill in the art will recognize that in other aspects, the flexible implant 900 could also be delivered and inserted into the disc space via other approaches which would then correct or alleviate spinal deformities in another anatomical plane or combination of anatomical planes by imparting the angled configuration of the implant wedge shape to the adjacent vertebral member in that orientation when the implant 900 is inserted and positioned between adjacent vertebral bodies in a selected intervertebral disc space. For example, if the flexible implant 900 is delivered and inserted into the disc space via a posterior approach, the implant would correct or alleviate coronal spinal deformities by imparting the angled configuration of the implant wedge shape to the adjacent vertebral member in a coronal orientation when the implant is inserted and positioned between adjacent vertebral bodies in a selected intervertebral disc space.

The flexible implant 900 of FIGS. 9A-9C comprises a multi-component flexible assembly comprising a leading end 905 pivotally connected to a trailing end 910, a first lateral side wall 906, and an opposite second lateral side wall 907. The flexible implant 900 comprises substantially a wedge shape or configuration in a lateral or side-to-side orientation, as best shown in FIG. 9B. The implant 900 has an overall side-to-side or lateral wedge shaped configuration between a first lateral side wall 906 and an opposite second lateral side wall 907. The implant's first lateral side wall 906 is the narrow or short part of the wedge and the implant's opposite second lateral side wall 907 is the wider or taller part of the wedge configuration. Inserting the wedge shaped flexible implant 900 in a selected disc space permits or enables a physician to impart the lateral angled configuration of the implant's wedge shape to the adjacent vertebral bodies to thereby correct, improve or stabilize the sagittal spacing, curvature, orientation or alignment of the selected disc space and in turn the overall sagittal spinal anatomy.

The flexible implant assembly 900 has a flexible section comprised of the leading end 905 pivotally connected to the trailing end 910 at a first hinge 912 to thereby permit rotational motion between the leading end 905 relative to the trailing end 910. The leading end 905 has a physical configuration adapted to facilitate or ease insertion of the flexible implant 900 into a disc space. In this aspect, the leading end 905 has a rounded shape nose 903. The rounded nose 903 can impart a distracting force to adjacent vertebrae as the flexible implant 900 travels or is inserted into the disc space. The leading end 905 is pivotally connected to the trailing end 910 to form a hinged multi-piece flexible implant 900, as shown in FIGS. 9A-9C. The flexible section is a pivoting connection 912 that permits or enables the flexible spinal implant 900 to pivot or articulate about the pivoting connection 912 such that the flexible spinal implant 900 can bend and articulate as may be needed to permit delivery and insertion of the flexible implant 900 in a lateral approach, including to enable the implant to sufficiently articulate in order to traverse an insertion channel bend.

The flexible spinal implant 900 further comprises an interior implant aperture 950 defined and formed by the pivotally connected leading end 905, first lateral side wall 906, opposite second lateral side wall 907 and trailing end 910. The interior implant aperture 950 can be filled or packed with graft material before or after insertion of the flexible implant 900 into the selected disc space. The trailing end 910 also comprises an implant grip or attachment aperture 935 situated at the proximal implant end 937 which enables the coupling of an instrument (not shown) to the flexible spinal implant 900. The attachment aperture 935 would enable an instrument to couple to the flexile spinal implant 900 and deliver the flexible implant 900 through an implant insertion channel (not shown) into a selected disc space via a lateral approach.

A front to back, FIG. 8B, and a lateral side-to-side, FIG. 9B, wedge shaped flexible implant 800 and 900 has been discussed above in relation to a flexible implant 800 and 900 which correct, improve or stabilize coronal or sagittal spinal deformities, respectively. In another embodiment, a hybrid wedge shape implant may have shape variations in both a front to back and a lateral side-to-side direction and orientation. For example in one case, the narrow or short part of the wedge may partially lie both in the leading end and a first lateral side wall, and the wider or taller part of the wedge may partially lie in both the trailing end and the second lateral side wall. In such a hybrid implant case, the implant wedge shape may be used to correct or alleviate sagittal and coronal spinal deformities by simultaneously imparting the implant's angled configuration to the adjacent vertebral member in a front to back and a lateral side-to-side manner when the implant is inserted and positioned between adjacent vertebral bodies at a selected intervertebral disc space. In this way, the angled configuration of the wedge shape implant can be imparted to the adjacent vertebral bodies to thereby simultaneously correct, improve or stabilize sagittal and coronal spacing, curvature, orientation or alignment of the selected disc space and overall spinal anatomy. Those of skill in the art will recognize that other shapes and configurations may instead be used for such a flexible implant, e.g., cubed, cone shaped, spherical, cylindrical, etc. The implant shape or configuration finally used will depend on patient anatomy, physician selection or requirements or clinical need.

The novel aspect to correct or alleviate sagittal and/or coronal spinal deformities discussed with respect to FIGS. 8A-9C are also contemplated for the flexible implants previously discussed in relation to FIGS. 1-7. Each of the implants disclosed in FIGS. 1-7 may be configured to have a front to back or lateral side-to-side wedge shaped configuration similar to those discussed above with respect to FIGS. 8A-9A to correct, improve or stabilize coronal or sagittal spinal deformities, respectively. And, alternatively, may have a hybrid wedge shape configuration with shape variations in both a front to back and a lateral side-to-side orientation. Those of skill in the art will recognize that the final shape or configuration of the flexible implant to be used to correct, alleviate or improve spinal deformities will depend on a patient's anatomy, and the requirements of a surgical procedure or a physician's needs or requirements.

FIG. 10 shows a side view of a flexible implant 1000 and locking mechanism 1050 according to another embodiment of the present disclosure. The flexible implant 1000 is a multi-component flexible assembly comprising a leading end 1005 pivotally connected to a trailing end 1010 and a locking mechanism 1050. The flexible implant assembly has a flexible section comprised of the leading end 1005 is pivotally connected to the trailing end 1010 at a first hinge 1012 to thereby permit rotational motion between the leading end 1005 relative to the trailing end 1010. The leading end 1005 is pivotally connected to the trailing end 1010 to form a hinged multi-piece flexible implant 1000. The leading end 1005 comprises a first hinge surface 1003 and a second hinge surface 1007 which are respectively paired to and complimentary to a third hinge surface 1013 and a fourth hinge surface 1017 on the trailing end 1010. The pivoting connection 1012 enables the flexible spinal implant 1000 to pivot or articulate about the pivoting connection 1012 and the first and second hinge surfaces 1003 and 1007 to respectively travel relative to the complimentarily paired third and fourth hinge surfaces 1013 and 1017 in a pivoting hinged arrangement.

The flexible implant 1000 is preferably inserted into the disc space in a straight or substantially straight orientation, as discussed previously with reference to FIGS. 1-9C. Once the implant is in place inside the disc space, it may be desirable to entirely lock the flexible implant 1000 so that the flexible implant's components are no longer flexible, pivotable or moveable. This aspect is accomplished via the locking mechanism 1050 which locks the flexible implant's pivoting hinges at the flexible section in place and prevents any rotation, pivoting or movement of the implant's flexible section hinges once the locking mechanism 1050 is engaged.

The locking mechanism 1050 comprises a locking member 1025, a leading end locking passage 1015, a trailing end locking passage 1020, and at least one pair of adjacently and opposed hinge surfaces between the leading end 1005 and trailing end 1010. The locking mechanism 1050 may be engaged when the leading end locking passage 1015 and trailing end locking passage 1020 are sufficiently and substantially aligned when the implant is inserted into the disc space and the implant has a substantially straight orientation within the disc space. The locking member 1025 is then delivered via an instrument (not shown) and positioned at least partially inside both of the aligned leading end locking passage 1015 and trailing end locking passage 1020, and positioned to span across at least one pair of complimentary hinge surfaces in the implant's flexible section. The locking member 1025 may be delivered and inserted via at least one locking passage 1015 and 1020 complimentary pair. The locking passages 1015 and 1020 must come in complimentary locking passage pairs 1015 so that a locking member 1025 is able to span across the corresponding pair of complimentary hinge surfaces in the implant's flexible section when the locking passages 1015 and 1020 are substantially aligned in order to engage the locking mechanism 1050. Those of skill in the art will recognize that more than one complimentary pair of locking passages may be used in an implant. For example, to increase the locking stability and locking redundancy in any one flexible implant depending on the needs or requirements of patient anatomy, a physician or clinical need. The complimentary pair of locking passages is preferably oriented and positioned in the flexible implant flexible section such that when the locking mechanism is engaged, the flexible implant will no longer be flexible, pivotable or moveable within the disc space. The complimentary pair of locking passages 1015 and 1020 can be oriented and positioned concentrically or adjacently to an implant attachment aperture 145 of the flexible implant 1000, or other orientations and positions which permit the flexible implant 100 to be placed in a locking engagement.

In FIG. 10, the complimentary pair of locking passages 1015 and 1020 is oriented and positioned adjacent or next to the implant attachment aperture 145 of the flexible implant 1000. The locking member 1025 is positioned to span across one pair of complimentary hinge surfaces in the implant's flexible section, the second and fourth hinge surfaces 1007 and 1017. In this arrangement, the locking member 1025 will obstruct or prevent pivoting or hinge motion between the second hinge surface 1007 and the fourth hinge surface 1017. This results in a locking engagement where the locking member 1025 prevents pivoting or hinge motion between the leading end 1005 and the trailing end 1010 to thereby lock the flexible implant 1000 in its straight or substantially straight orientation in the disc space. Alternatively, the locking mechanism 1050 could be engaged by placing or positioning the locking member 125 to span across the first hinge surface 1003 and the fourth hinge surface 1013 of the implant's flexible section.

In another embodiment, the locking mechanism 1050 may have a longer length such that it can simultaneously span across both paired hinge surfaces of the implant's flexible section, i.e., the first and third hinge surfaces 1003 & 1013 and the second and fourth hinge surfaces 1007 and 1017. Those of skill in the art will recognize that the locking mechanism 1050 contemplated herein will have a locking member 10025 which is positioned to span across a sufficient number of pairs of complimentary hinge surfaces of the implant's flexible section to lock the flexible implant 1000 in a straight or substantially straight orientation within the disc space. The locking mechanism 1025 may also comprise more than one set of locking member 1025 and locking passages 1015 and 1020 depending on the surgeon's preference or need, or the requirements or needs of the medical procedure. For example, in the case of the flexible implant 100 shown in FIG. 5A, a locking mechanism could be used and engaged by placing or positioning a locking member in appropriate locking passages to span across paired hinge surfaces of the implant's flexible section which correspond to pivoting connections 112, 117 and 127 and/or pivoting connections 112, 115 and 130 to lock the flexible implant 100 in a straight or substantially straight orientation within the disc space. In another case, such as the flexible implant 200 shown in FIG. 5C, a locking mechanism be could used and engaged by placing or positioning a locking member in appropriate locking passages to span across paired hinge surfaces of the implant's flexible section which correspond to pivoting connections 212 and 217 and/or pivoting connections 207 and 223 to lock the flexible implant 200 in a straight or substantially straight orientation within the disc space.

In some cases, it may be desirable to only partial lock the flexible implant. In such a case, the locking member may be positioned to span across paired hinge surfaces of the implant's flexible section which are less than the corresponding pivoting connections resulting in not all hinged surfaces being obstructed by the locking member, thereby leaving the flexible implant partially locked and partially moveable. For example in the flexible implant 100 shown in FIG. 5A, a locking mechanism could be partially engaged by placing or positioning a locking member in appropriate locking passages to span across less than all the paired hinge surfaces of the implant's flexible section which correspond to pivoting connections 112, 117 and 127 or pivoting connections 112, 115 and 130 to partially lock the flexible implant 100 within the disc space. And, in the flexible implant 200 shown in FIG. 5C, a locking mechanism could be partially engaged by placing or positioning a locking member in appropriate locking passages to span across less than all paired hinge surfaces of the implant's flexible section which correspond to pivoting connections 212 and 217 or pivoting connections 207 and 223 to partially lock the flexible implant 200 within the disc space.

The locking member 1025, the leading end locking passage 1015 and the trailing end locking passage 1020 preferably have complimentary configurations so that the locking member 25 can be delivered and inserted into the aligned leading end locking passage 1015 and trailing end locking passage 1020 to engage the locking mechanism 1050. As noted above, the leading end locking channel 1015 and the trailing end locking channel 1020 may be located concentrically or adjacent to an implant attachment aperture 145 to permit the locking member 1025 to be delivered once the spinal implant has been delivered and inserted in place. The locking member 1025 will cooperate with at least one pair of complimentary leading end locking channel 1015 and trailing end locking channel 1020 to permit the locking mechanism to be engaged.

The locking member 1025 is preferably configured and sized so as to be capable of being delivered through the same implant insertion channel that is used to deliver the flexible implant. For example, similar to an implant insertion channel 167 like that disclosed in FIG. 5B for delivery of the flexible implant 100. Using the same implant insertion channel to deliver the locking member 1025 can minimize the number of delivery instruments or devices and thereby reduce the number of components used in a surgical procedure. However, other insertion channels or devices may be used instead to deliver the locking member 1025 depending on a physician or patient anatomy, or the needs or requirements of a medical procedure. The locking member 1025 will be firmly held in place in a final locking position once delivered and positioned in the leading end locking passage 1015 and trailing end locking passage 1020.

The locking member 1025 may be sized to enable it to be delivered and positioned in the flexible implant in a locking position or locking engagement position. The locking member 1025 may be sized such that it will firmly engage the leading end locking passage 1015 and trailing end locking passage 1020 via a friction fit or interference fit in its final locking position. Those of skill in the art will recognize that the locking member 1025 may have other shapes, size, length or configurations such that it can travel in the leading end locking passage 1015 and trailing end locking passage 1020 and also be held via a friction fit or interference fit in the leading end locking passage 1015 and trailing end locking passage 1020. The locking member 1025 may also be configured to firmly engage the leading end locking passage 1015 and trailing end locking passage 1020 via other securing mechanisms, for example, via a snap fit.

The locking member 1025 may be manufactured to have desired or needed characteristics or properties which enable it to be delivered and positioned in the flexible implant in a locking position or locking engagement position. The locking member 1025 should be of sufficient length to, when inserted in the locking passages, span across at least one pair of hinge surfaces of an implant's flexible section which correspond to pivoting connections to prevent and obstruct any further hinge movement and lock the flexible implant 1000 in a substantially straight orientation within the disc space. The locking member should also be manufactured with physical properties where it is sufficiently strong enough to perform the locking function without shearing or breaking once it is positioned in the locking passages 1015 and 1025.

The locking member 1025 may also have physical properties and characteristics which permit it to travel and traverse the implant delivery path and implant delivery components, such as an insertion channel. In a preferred embodiment, the locking member 1025 is manufactured to have characteristics or properties which permit it to travel and traverse the implant delivery path and the implant insertion channel used to deliver the flexible implant, such as the implant insertion channel 167 disclosed in FIG. 5B for delivery of the flexible implant 100. Using a similar implant insertion channel to deliver the locking member 1025 would be advantageous and efficient since such an approach reduces the number of delivery instruments, devices and components used in a surgical procedure.

During locking member 1025 delivery, the locking member 1025 would travel inside an implant insertion channel similar to insertion channel 160 to reach the desired or selected disc space level. In order for the locking member 1025 to reach and enter the disc space in a substantially straight lateral approach orientation, the locking member 1025 will be guided, by an instrument (not shown) to travel through the interior walls of the implant insertion channel. The locking member 1025 will reach and have to traverse a channel turn section similar to the channel turn section 164 of FIG. 5B. The locking member 1025 must be of appropriate length so that as the locking member 1025 continues to travel into the channel turn section, the locking member 1025 will able to travel through and around the corner of the channel turn section. Once the locking member 1025 travels past the channel turn section, it can be reoriented to approach the flexible implant in the disc space in a substantially straight lateral approach orientation as it exits the channel turn section and approached the flexible implant. In this manner, the locking member 1025 can turn from its delivery path and enter the disc space in a substantially straight lateral approach orientation where it can be delivered to the aligned leading end locking passage 1015 and the trailing end locking passage 1020 of the inserted flexible implant to engage the locking mechanism 1050.

In some instances, the locking member 1025 may also have physical properties which permit it to flex, deflect, bend or deform as it travels and traverses the bend or corner of an implant insertion channel on its way to the implant locking passages 1015 and 1020. For example, in a case of a flexible implant with many pivoting or movable hinges at its implant flexible section, it may be desirable to have a locking member 1025 that is longer in length and that thus may not be able to traverse the insertion channel turn section due to its length. In such an instance, the locking member 1025 will have physical properties and characteristics which permit it to flex, deflect, bend or deform sufficiently and as needed to enable the locking member 1025 to traverse the insertion channel turn section. In particular, locking member 1025 would be manufactured such that it can flex, deflect, bend or deform sufficiently to enable it to travel and traverse through and around the channel turn section of the insertion channel and into the implant's aligned locking passages 1015 and 1020.

During delivery of such a bending or deforming locking member 1025, the locking member 1025 would travel inside the implant insertion channel to reach the selected disc space level. In order for the locking member 1025 to reach and enter the disc space in a substantially straight lateral approach orientation, the locking member 1025 will be guided by the interior walls of the implant insertion channel 1160. The deforming locking member 1025 will reach and have to traverse a channel turn section. The channel turn section will interact with and force the locking member 1025 to flex, deflect, bend or deform while traveling through the channel turn section. This cannel turn section interaction imparts a force to deform the locking member 1025 such that the locking member 1025 flexes, deflects, bends or deforms to thereby enable travel of the locking member 1025 through the channel turn section 1164. In this manner, the locking member 1025 is adapted to flex, deflect, bend or deform and turn from its delivery path and enter the disc space in a substantially straight lateral approach orientation. The forced flexing, deflection, bending or deforming by the channel turn section interaction transitions and assists the locking member 1025 to travel and traverse through the channel turn section of the insertion channel and into the implant's aligned locking passages 1015 and 1020.

The locking member 1025 and corresponding locking passages may also be manufactured to have a desired or needed configurations which enables it to be delivered and positioned in the flexible implant in a locking position or locking engagement position. In one embodiment, shown in FIG. 10, the leading end passage 1015 and trailing end locking passage 1020 have cylindrical configurations which sufficiently and substantially align when the implant is inserted into the disc space and the implant takes on a straight or substantially straight orientation within the disc space. The locking member 1025 will in turn have a complimentary cylindrical rod or pin configuration which is adapted to be inserted into the substantially aligned leading end passage 1015 and trailing end locking passage 1020. The locking member 1025 will also have an appropriate size and length so as to be able to be positioned to span across the second and fourth hinge surfaces 1007 and 1017.

Those of skill in the art will recognize that other complimentary shapes, size and configurations may be used for the locking member 1025, the leading end locking passage 1015 and the trailing end locking passage 1020 so long as these components have complimentary configurations that permit the locking member 1025 to be inserted into and to traverse the leading end locking passage 1015 and trailing end locking passage 1020 to reach a locking position. For example, in one case, the locking member 1025, leading end locking passage 1015 and trailing end locking passage 1020 may have complimentary T-configurations. In this case, the leading end locking passage 1015 and trailing end locking passage 1020 would have a T-shaped passage cross-section and the locking member 1025 would have a complimentary T-shaped rail cross-section which can be inserted and can travel therein.

A flexible implant with an engagable locking mechanism discussed with respect to FIG. 10 is also contemplated for the flexible implants previously discussed in relation to FIGS. 3-4 and 6A-7. Each of the implants disclosed in FIGS. 3-4 and 6A-7 may be configured to include a locking mechanism which comprises a locking member and one or more leading and trailing end locking passages with complimentary configurations such that the locking member can be delivered and inserted into appropriately aligned leading and trailing end locking passages to engage the locking mechanism and thereby lock the flexible implant such that it is no longer be flexible, pivotable or moveable within the disc space. The locking mechanism, i.e., locking member and leading and trailing end locking passages, may be located concentrically or adjacent to an implant attachment aperture to permit the locking member to be delivered once the spinal implant inserted in the disc space. With reference to FIGS. 3-4 and 6A-7, the locking mechanism can be used with the disclosed flexible implants 30, 60, 250, 260, 270, 300, 400, 410, and 420 where the locking mechanism is positioned across the flexible sections 34, 64, 254, 264, 274, 310, 404, 414, 424. This aspect permits the flexible sections 34, 64, 254, 264, 274, 310, 404, 414, 424 to be locked when the locking member is delivered and inserted into appropriately aligned leading and trailing end locking passages to engage the locking mechanism and thereby lock the flexible implant 30, 60, 250, 260, 270, 300, 400, 410, and 420 such that flexible implant will no longer be flexible, rotatable, pivotable or moveable within the disc space.

In a further aspect contemplated for the flexible implants depicted and discussed with respect to FIGS. 1-10, the flexible implant may be manufactured to have properties or characteristics such that the flexible section can or will become rigid or substantially rigid once the implant is fully implanted in the disc space. The flexible section can be manufactured to have properties or characteristics which enable the implant become rigid at a threshold temperature, a patient's body temperature, a desired time after implant insertion or over time after implant insertion. For example, in one case, as soon as the implant is inserted in the disc space, over a desired or predetermined time period as the fusion is setting. The flexible implant once rigid would thereafter no longer maintain implant flexibility or pliancy. In one aspect, the implant rigidity characteristic may be provided through the use of shape memory nitinol, other shape memory materials or other curable substance, such as an epoxy, poly methyl methacrylate (PMMA) or a polycarbonate, which can or would be adapted to reach rigidity in a patient anatomical environment. This aspect or property may be used where desired or required by a patient's anatomy, a surgical procedure or a physician's requirement.

The flexible implants and locking member disclosed in this disclosure are preferably comprised of biocompatible materials substrates which can be attached to the novel flexible implant sections to form a whole flexible spinal implant or locking member. The biocompatible material substrate may include, among others, polyetheretherketone (PEEK) polymer material, homopolymers, co-polymers and oligomers of polyhydroxy acids, polyesters, polyorthoesters, polyanhydrides, polydioxanone, polydioxanediones, polyesteramides, polyaminoacids, polyamides, polycarbonates, polylactide, polyglycolide, tyrosine-derived polycarbonate, polyanhydride, polyorthoester, polyphosphazene, polyethylene, polyester, polyvinyl alcohol, polyacrylonitrile, polyamide, polytetrafluorethylene, poly-paraphenylene terephthalamide, polyetherketoneketone (PEKK); polyaryletherketones (PAEK), cellulose, carbon fiber reinforced composite, and mixtures thereof. The biocompatible material substrate may also be a metallic material and may include, among others, stainless steel, titanium, nitinol, platinum, tungsten, silver, palladium, cobalt chrome alloys, shape memory nitinol and mixtures thereof. The biocompatible material used can depend on the patient's need and physician requirements.

While embodiments of the invention have been illustrated and described in detail in the present disclosure, the disclosure is to be considered as illustrative and not restrictive in character. All changes and modifications that come within the spirit of the invention are desired to be protected and are to be considered within the scope of the disclosure.

1. A spinal implant for insertion into an intervertebral disc space, the implant comprising:
a leading end;
a trailing end flexibly connected to the leading end; and
a locking mechanism adapted to lockingly engage the spinal implant to prevent motion between the leading end and trailing end;
wherein the implant is deformable about a flexible section to thereby permit a substantially straight entry of the implant into a selected disc space.

2. The spinal implant of claim 1, wherein the locking mechanism comprises
a leading end locking passage,
a trailing end locking passage which substantially aligns with the leading end locking passage when the implant is inserted in the selected disc space, and
a locking member adapted to be inserted into the leading end locking passage and trailing end locking passage;
wherein the locking mechanism is engaged when the locking member spans the flexible section at a pivotal connection and is at least partially inserted into both the substantially aligned leading end locking passage and trailing end locking passage thereby preventing motion between the leading end and the trailing end.

3. The spinal implant of claim 2, wherein the locking member is secured in both the aligned leading end locking passage and trailing end locking passage through a friction fit or interference fit.

4. The spinal implant of claim 2, wherein the locking member is adapted to cooperatively deform to facilitate insertion of the locking member into the aligned leading end locking passage and trailing end locking passage.

5. The spinal implant of claim 2, wherein the leading end locking passage, the trailing end and the locking member have complimentary and cooperative configurations to enable the locking member to be inserted into the aligned leading end locking passage and trailing end locking passage.

6. The spinal implant of claim 1, wherein the leading end and trailing end form a front to back wedge configuration.

7. The spinal implant of claim 6, wherein the front to back wedge configuration alleviates coronal spinal deformity when the spinal implant is inserted in the selected disc space.

8. The spinal implant of claim 1, wherein the leading end and trailing end form a lateral side-to-side wedge configuration.

9. The spinal implant of claim 8, wherein the lateral side-to-side wedge configuration alleviates sagittal spinal deformity when the spinal implant is inserted in the selected disc space.

10. An implant system for insertion into an intervertebral disc space, the implant comprising:
a spinal implant deformable about a flexible section to thereby permit a substantially straight entry of the implant into a selected disc space, the spinal implant comprising
a leading end including a leading end locking passage,
a trailing end flexibly connected to the leading end, and having a trailing end locking passage which substantially aligns with the leading end locking passage when the implant is inserted into a selected disc space; and
a locking member adapted to be inserted into the leading end locking passage and trailing end locking passage to lockingly engage the spinal implant to prevent motion between the leading end and trailing end;
wherein, in locking engagement, the locking member spans the flexible section at a pivotal connection and is at least partially inserted into both the substantially aligned leading end locking passage and trailing end locking passage thereby preventing motion between the leading end and the trailing end.

11. The implant system of claim 10, wherein the locking member is secured in both the aligned leading end locking passage and trailing end locking passage through a friction fit or interference fit.

12. The implant system of claim 10, wherein the locking member is adapted to cooperatively deform to facilitate insertion of the locking member into the aligned leading end locking passage and trailing end locking passage.

13. The implant system of claim 10, wherein the leading end and trailing end form a front to back wedge configuration.

14. The implant system of claim 13, wherein the front to back wedge configuration alleviates coronal spinal deformity when the spinal implant is inserted in the selected disc space.

15. The implant system of claim 10, wherein the leading end and trailing end form a lateral side-to-side wedge configuration.

16. The implant system of claim 15, wherein the lateral side-to-side wedge configuration alleviates sagittal spinal deformity when the spinal implant is inserted in the selected disc space.

17. A spinal implant for insertion into an intervertebral disc space, the implant comprising:
a leading end;
a trailing end flexibly connected to the leading end, wherein the implant is deformable about a flexible section between the leading end and trailing end to thereby permit a substantially straight entry of the implant into a selected disc space; and
a locking member adapted to lockingly engage the leading end and trailing end via a friction fit or interference fit;
wherein the locking member is locking engaged by spanning the flexible section at a pivotal connection between the leading end and trailing end thereby prevent motion between the leading end and the trailing end.

18. The spinal implant of claim 17, wherein the leading end and trailing end form a front to back wedge configuration which is adapted to alleviate coronal spinal deformity when the spinal implant is inserted in the selected disc space.

19. The spinal implant of claim 17, wherein the leading end and trailing end form a lateral side-to-side wedge configuration which is adapted to alleviate sagittal spinal deformity when the spinal implant is inserted in the selected disc space.
